(19) United States
(12) Patent Application Publication
(10) Pub. No.: US 2022/0118070 A1
(43) Pub. Date: Apr. 21, 2022

(54) CANCER THERAPY BY MODIFYING NEOANTIGEN EXPRESSION

(71) Applicant: Nicholas LA THANGUE, Oxford Oxfordshire (GB)

(72) Inventor: Nicholas LA THANGUE, Oxford Oxfordshire (GB)

(21) Appl. No.: 17/605,713

(22) PCT Filed: Apr. 24, 2020
(86) PCT No.: PCT/GB2020/051023

§ 371 Date: Oct. 22, 2021

(30) Foreign Application Priority Data
Apr. 25, 2019 (GB) 1905780.1

(51) Int. Cl. A61K 39/00; C12Q 1/6886; G01N 33/50; A61K 35/17; A61K 45/06; C07K 14/705

(52) U.S. Cl. CPC A61K 39/0011; C12Q 1/6886; G01N 33/5011; C12Q 2600/106; A61K 45/06; C07K 14/705; A61K 35/17

(57) ABSTRACT

This invention relates to methods, agents and compositions for regulating the neoantigen landscape of cancer cells, based on the realisation that intron retention in a cancer cell can be manipulated. Agents that can modify retained intron neoantigen expression in cancer cells are described, and their use in drug discovery and therapy. One aspect provides an agent for use in a method of treating cancer by modifying the neoantigen profile of at least one cancer cell. The agent may typically be a protein arginine N-methyltransferase 5 (PRMT5) inhibitor, and/or may reduce methylation of an E2F protein, for example E2F1.

### FIELD OF THE INVENTION

This invention relates to methods, agents and compositions for regulating the neo-antigen landscape of cancer cells. In particular, the invention relates to the identification of agents that can modify retained intron (RI) neo-antigen expression in cancer cells, and their use in drug discovery and therapy.

### BACKGROUND OF THE INVENTION

Cancers use a diverse array of mechanisms to evade the immune system such as down-regulating immune checkpoint pathways. The development of therapeutic antibodies targeting immune checkpoints, such as CTLA-4, anti-PD-1 and PD-L1, represents one of the most important breakthroughs in cancer therapy in the past decade (Schumacher T. N. et al. 2015). It is widely accepted that tumors with more mutations likely generate more neo-antigens, which can be recognized by tumor infiltrating T cells. It has been shown that checkpoint blocking antibodies reactivate these T cells in vivo and induce tumor regressions. As a result, cancers with high mutation rates, such as melanoma and lung cancer, are more susceptible to checkpoint blockade therapies (Tran E. et al. 2015; Y-C Lu et al. 2016). However, cures with checkpoint inhibitors remain rare and many cancer patients experience modest clinical benefit. Therefore, cancer immunotherapy requires new approaches to drive potent tumour-specific immune responses that provide clinical benefit to large numbers of patients.

Arguably a better immuno-therapeutic approach which has broad application, including cancers which are unresponsive to checkpoint inhibitors, is based on understanding the neo-antigen landscape of a cancer. Neo-antigens have been validated in cancer patients as critical T cell targets where they strengthen the immune response against tumours by stimulating the adaptive immune response (Linnemann C. et al. 2015). Tumour-specific neo-antigens originate in part from non-self peptides, such as arising from oncogenic somatic mutations. Because they are not present on normal tissues they bypass central thymic tolerance when presented to the immune system in complex with HLA. However, the identification of neo-antigens presents a key therapeutic challenge; machine learning algorithms that predict high affinity HLA peptides, together with massively parallel sequencing to detect tumour mutations, has had some benefit (Yadav M. et al. 2014; Cohen C. J. et al. 2015; Bassani-Sternberg M. et al. 2016; Freudenmann L. K. et al. 2018). Further, tumour genomes vary enormously in the mutational burden; some have hundreds of mutations where only a minority result in true tumour-specific neo-antigen presentation, and others have a low mutational burden, making neo-antigens challenging to identify (Y-C Lu et al. 2016).

New and improved cancer therapies are still needed.

### SUMMARY OF THE INVENTION

The present invention is based on surprising cancer biology results that highlight RNA splicing as a potential source of neo-antigens. In particular, the invention relates to the realisation that intron retention in a cancer cell can be manipulated. This unexpected insight provides an opportunity to regulate the neo-antigen landscape and allows for the development of a new type of cancer vaccine. This can be used, for example, to develop either disease-specific or patient-specific vaccines.

Without being bound by theory, the identification of these new effects and new activities provides new opportunities to target and treat cancer cells in vivo.

An agent that regulates the neoantigen landscape of the cancer may be used as a therapeutic agent itself, where it can improve the presentation of neoantigens on cancer cells when administered to the patient and thereby enhance an immune response against the cancer. In this approach, the agent may advantageously be part of a combination therapy with an immunotherapy, such as a checkpoint inhibitor, that enhances the immune response against the cancer cell. In an alternative approach, an agent that regulates the neoantigen landscape of a cancer can be used to manipulate a patient's cancer cells, either in vivo or in vitro, to allow the neoepitope repertoire of the cancer to be interrogated and/or to enhance the expression of neoepitopes within that cancer. Once the neoepitopes present on the cancer cells are identified, a cancer vaccine comprising those neoepitope(s) can be prepared. Accordingly, a vaccine comprising identified neoepitopes may be prepared to be specific for the individual patient, or for a particular cancer type or subtype where the neoepitopes may be conserved across patients.

The unexpected finding that the neoepitopes present in a cancer cell can be stabilised allows for the identification of neoantigens with therapeutic utility. Typically, cancer cells from a patient or a population of patients can be analysed to identify RNAs containing alterative splicing, in particular retained introns. Optionally the patient is treated with the agent prior to sampling the cancer cells (or otherwise obtaining RNA transcript information from the cancer cells), for example with an agent such as an E2F methylation inhibitor, optionally a PRMT5 inhibitor. In this way, the patient's cancer cells in vivo can be effectively forced to express neoepitopes that then expose the cancer cells to the immune system thereby targeting them for destruction.

An alternative to administering the agent to the patient, is to sample cancer cells from the patient and treat them with the agent after they have been sampled (e.g. in vitro), to regulate the presence of RI-containing RNAs. Neoepitopes encoded by the RI-containing RNAs can then be determined.

The identified neoepitopes can be provided as a therapeutic composition suitable for administration, typically as a personalised therapy to the patient from whom the RNA splicing information was obtained. In this way, a personalised cancer vaccine can be provided.

The total number of retained introns can increase or decrease in treated cancer cells, as observed in the Examples. Namely, there can be a shift from ‘normal’ neo-antigens expressed on cancer cells (which are recognised as self and therefore do not break tolerance) to new (up-regulated RIs) which provide non-self recognition and potential as cancer vaccines. The invention therefore allows for a re-balancing of the RI and neoantigen profile, wherein RI neoantigens that are not recognised as non-self (i.e. are tolerated) are reduced, and typically accompanied by a concomitant increase in the amount of, or relative visibility to the immune system of, neoantigens that are recognised as non-self.

In a first aspect, the invention provides an agent for use in a method of treating cancer by modifying the neoantigen profile of at least one cancer cell. In a related aspect, the invention provides an agent for use in the manufacture of a medicament for the treatment of cancer by modifying the neoantigen profile of at least one cancer cell.

In a second aspect, the invention provides a method of treating a cancer in a patient, comprising treating the patient with an agent that modifies the neoantigen profile of the cancer.

In certain embodiments, the number of neoantigens present on the cancer cell is increased by contact with the agent. In other embodiments, the ability of the cancer cell and/or neoantigen to be recognised by the patient's immune system, for example by T cell receptors, is increased.

This may be by increasing the expression of neoepitopes in or on the cancer cell, that can be presented for example by MHC-I and be recognised by a T cell receptor on CD8+ CTLs leading to destruction of the cancer cell. The neoepitope is typically one that binds to MHC, usually MHC-I. Strong binding to MHC-I will be helpful in the cell being recognised as defective (i.e. containing non-natural proteins) and being recognised for destruction by CD8+ T cells (CTLs). The agent may in some embodiments increase intron retention in a cancer cell or increase the stability of one or more retained introns in a cancer cell. In some embodiments, the neoantigens comprise one or more retained introns and are presented by the cancer cell following contact with the agent.

The evidence in the Examples shows that manipulating methylation of certain proteins in cancer cells makes it possible to regulate the production of stable retained introns in those cancer cells.

Therefore, in certain embodiments, the agent alters the methylation state of a transcription regulator, typically the level of arginine methylation. The transcription regulator may typically be an E2F protein such as E2F1, and in some embodiments the agent may thus be an inhibitor of E2F1 methylation. The agent may modulate the ability of a tudor domain protein, which in some embodiments may be the p100/TSN tudor domain protein, to bind to a transcription factor when the transcription factor is bound to chromatin.

The agent may typically be a protein arginine N-methyltransferase 5 (PRMT5) inhibitor, and/or may reduce methylation of an E2F protein, for Example E2F1. A PRMT5 inhibitor reduces the expression and/or activity of the enzyme PRMT5. In particular a PRMT5 inhibitor typically reduces or abolishes the ability of PRMT5 to methylate E2F1 protein.

The presence of RIs and their potential impact on non-self, and therefore neo-antigens, may be relevant for all cancer drugs and therapies that have cancer cell intrinsic effects, including radiation. The inventor happens to have discovered this effect through work on the PRMT5/E2F axis, but the invention is not so limited. The finding that “traditional” cancer-killing therapeutic agents, such as chemotherapeutics and radiation, not only have a cytotoxic effect but also modulate neoantigen presentation on the cancer cells, provides a new treatment opportunity that links the mechanism of these traditional therapies with newer immunotherapies. Any such agent can be used according to the invention. In some embodiments, the agent is cytotoxic. In some embodiments, the agent affects transcription within the cancer cell and/or affects epigenetics within the cancer cell. In some embodiments, the agent has an impact on transcription and/or gene expression in the cancer cell, of which non-limiting examples include Histone Deacetylase (HDAC) inhibitors and ionising radiation (which damages the DNA and so affects transcription and gene expression). In some embodiments, the agent targets chromatin or targets chromatin remodelling. Examples of approved epigenetic drugs include the DNA methyltransferase 1 (DNMT1) inhibitor 5-azacitidine and the histone deacetylase (HDAC) inhibitor vorinostat.

The inventor has observed experimentally that the retained intron effect is provided by HDAC inhibitors (data not shown).

The agent may be, comprise or consist of, an antisense molecule, an shRNA, an siRNA, a small molecule with a molecular weight less than 750 Da, a protein, an antibody or an antibody fragment. In some embodiments, the antibody or antigen-binding fragment thereof is human or humanised. In some embodiments, the agent can be or comprise radiation, typically ionising radiation, or a source of that radiation. Radiotherapy of cancer is well known. Examples of radiation for use in cancer therapy are external radiotherapies such as X-rays, gamma rays and proton beam therapies. Radiation can also be provided by an internal radiotherapy, which may be brachytherapy or the use of an unsealed radionuclide. Radionuclides for use in brachytherapy include Cesium-231, Cesium-137, Cobalt-60, Iridium-192, Iodine-125, Palladium-103, Ruthenium-106 and Radium-226. Radionuclides typically used for unsealed therapies include Iodine-131, I-MIBG, Phosphorus-32 and Yttrium-90. Antibody-radionuclide conjugates may also be used.

The agent may in some embodiments be an E2F1 protein that is arginine-methylation defective. The agent may be an E2F1 protein with a mutation at positions R111 and/or R113 (for example R111K and/or R113K), which are defective in PRMT5 methylation.

In some embodiments, the agent may be an siRNA molecule that reduces expression of PRMT5.

In some embodiments, the therapy may be effected by a method of treatment that comprises the steps of:
(a) administering the agent to the cancer patient;
(b) identifying retained introns in RNA transcripts from the cancer patient to whom the agent has been administered, for example from a tumour biopsy obtained from the patient;
(c) identifying one more neo-antigens that result from the retained introns.

This method can optionally contain one or more steps, such as producing proteins or peptides comprising the identified neoantigens, formulating the identified neoantigen(s) into a pharmaceutical composition and/or administering to a patient one or more of the identified neoantigens.

The agent that stabilises the retained introns can be provided as part of a combination therapy, for example in combination with a chemotherapeutic agent or an immunotherapy. The neoepitopes that are identified can also be provided as a combination therapy, for example in combination with a chemotherapeutic agent or an immunotherapy. The immunotherapy may optionally be (i) a checkpoint inhibitor optionally selected from: an anti-PD-1 antibody, an anti-PD-L1 antibody, an anti-CTLA-4 antibody, an anti-LAG-3 antibody, or an anti-CTLA-4 antibody; or (ii) a CAR-T or CAR-NK cell. Suitable antibodies and CAR-cells are known in the art, for example nivolumab (anti-PD-1), pembrolizumab (anti-PD-1), ipilumab (anti-CTLA-4), atezolizumab (anti-PD-L1), avelumab (anti-PD-L1), durvalumab (anti-PD-L1) and cemiplimab (anti-PD-1).

The combination therapy may in some embodiments comprise an oncolytic virus. Oncolytic viruses lyse tumour cells and thereby release tumour antigens and neoantigens. This release of tumour antigens and neoantigens can enhance the immune response to the tumour. So, the agent or neoepitope therapy of the invention may enhance the neoepitope-releasing effect of an oncolytic virus.

The combination therapy may comprise the agent that stabilises the retained introns and the identified neoepitopes, in some embodiments.

In a further aspect, the invention provides a method of determining whether a patient will respond favourably to cancer immunotherapy, comprising
(a) administering to the cancer patient an agent able to regulate the neoantigen landscape of the cancer; and
(b) identifying the patient as likely to benefit from immunotherapy if one or more introns are identified in RNA transcripts, optionally from a tumour biopsy, obtained from the cancer patient to whom the agent has been administered.

In yet another aspect, the invention provides an in vitro method for identifying an agent useful in treating cancer, comprising contacting or incubating the agent with a cell and assessing whether the level of retained introns in RNA transcripts increases following contact, wherein an increase in retained introns indicates that the agent is useful in treating cancer.

The invention also provides a method for identifying an agent able to regulate the neoepitope landscape of a cancer cell, wherein the agent is contacted or incubated with a cell and one, two or all three of the following features, alone or in combination, are detected following contact:
Change, for example increase, in RNA splicing;
Change, for example increase, in retained introns;
Change, for example decrease, in the interaction between p100/TSN and E2F1.

In a further aspect, the invention provides an in vitro method of increasing neoepitope expression in a cell, comprising contacting or incubating the cell with an agent that increases intron retention in a cell, optionally wherein the agent that increases intron retention in a cell alters (e.g. reduces or inhibits) the methylation state of an E2F protein.

Another aspect provides an in vitro method for identifying a neoantigen for use in treating cancer, comprising the steps of:
(a) identifying retained introns in RNA transcripts from a cancer cell from a patient;
(b) identifying one more neo-antigens expressed by the retained introns.

The method of this aspect may further comprising the step of producing the one or more neo-antigens and optionally formulating the one or more neo-antigens into a pharmaceutical composition. The cancer cell in step (a) may be from a patient to whom an agent has been administered to modify the cancer neoantigen profile.

Another aspect of the invention provides a pharmaceutical composition comprising an agent capable of modifying the neoantigen profile of a cancer cell.

The invention also provides, in another aspect, a pharmaceutical composition comprising one or more neoantigens, wherein the one or more neoantigens are encoded by RNA comprising one or more retained introns. These neoantigens are typically identified using an agent or method according to the invention. This composition may be prepared for a pre-determined patient or cancer, typically a patient that has been identified to have a cancer expressing that neoepitope. The patient may have been treated with an agent to regulate the expression of the neoepitopes.

A pharmaceutical composition will typically contain at least one pharmaceutically-acceptable excipient or diluent. A pharmaceutical compositions for use as a vaccine, for example a cancer vaccine, comprising neoepitopes may optionally contain an adjuvant.

According to another aspect, the invention provides an ex vivo composition comprising RNA sequences encoding one or more neoantigens, wherein each RNA sequence comprises at least one retained intron per neoantigen. The RNA sequences can in some embodiments be human and the composition can optionally comprise one or more non-human reagents, such as a non-human DNA or RNA polymerase molecule.

The invention also provides, in another aspect, an immune cell engineered to express one or more receptors that binds, e.g. specifically binds, to one or more neoantigens encoded by RNA containing at least one retained intron. The one or more neoantigens are typically identified using an agent or method according to the invention. Examples of immune cells include a T cell (in which case the one or more receptors may be a T cell receptor, or an engineered version thereof) or a Natural Killer cell, optionally a CAR-T cell or a CAR-NK cell. A population of such immune cells is also provided, optionally comprising at least two different cells with affinity for different neoantigens.

The invention also provides an autologous cell therapy product comprising an expanded T cell population having affinity for a neoantigen present on a cancer cell, wherein the population was expanded from one or more T cells sampled from a tumour microenvironment.

### BRIEF DESCRIPTION OF THE DRAWINGS

FIG. 1A: Retained introns in E2F-target genes contribute to the neo-antigen landscape of cancer cells. A) Arginine methylation of E2F by PRMT5 connects its transcriptional regulation activity with that of alternative splicing (AS) regulation. B) Schematic of personalised neoantigen therapy approach with optional PRMT5 inhibitor treatment.

FIG. 1B: E2F and PRMT5 activity influences intron retention.

A) RNA-seq data from wild-type E2F expressing HCT116 colon carcinoma cell lines treated with PRMT5 inhibitors was analysed for statistically significant (FDR<0.01) changes in alternative splicing events of E2F-target genes. The number of intron retention events observed to be significantly up-regulated or down-regulated, as compared to untreated cells, is displayed.

B) Wild-type E2F expressing HCT116 cells were treated with PRMT5 inhibitor as per the conditions used for the RNA-seq. Total RNA was isolated and qRT-PCR was performed using primers to detect the retention of intron 8 of the CDK10 gene. An immunoblot is included to indicate protein levels. A blot with antisymmetric di-methyl antibody (SDme) was performed as a control to confirm activity of the PRMT5 inhibitor.

C) TCGA datasets from colon adenocarcinoma (COAD), eosophageal carcinoma (ESCA), liver hepatocellular carcinoma (LIHC), pancreatic adenocarcinoma (PAAD) and stomach adenocarcinoma (STAD) were examined for the presence of a retained intron 8 in the CDK10 gene using TCGA SpliceSeq software (MD Anderson Cancer Center; https://bioinformatics.mdanderson.org/TCGASpliceSeq.index.jsp). Displayed are the percent spliced-in values from tumour and adjacent normal samples.

D) A neo-antigen prediction algorithm was performed on the RNA-seq data from wild-type E2F expressing HCT116 cells treated with PRMT5 inhibitors to determine potential RI-derived neoantigens that are predicted to have strong affinity for HLA. Displayed are the numbers of unique and shared RI-derived antigens, as compared to untreated cells. The algorithm used to predict the neo-antigen peptides is as described by Smart et al, Nature Biotechnology Volume 36, pages 1056-1058 (2018).

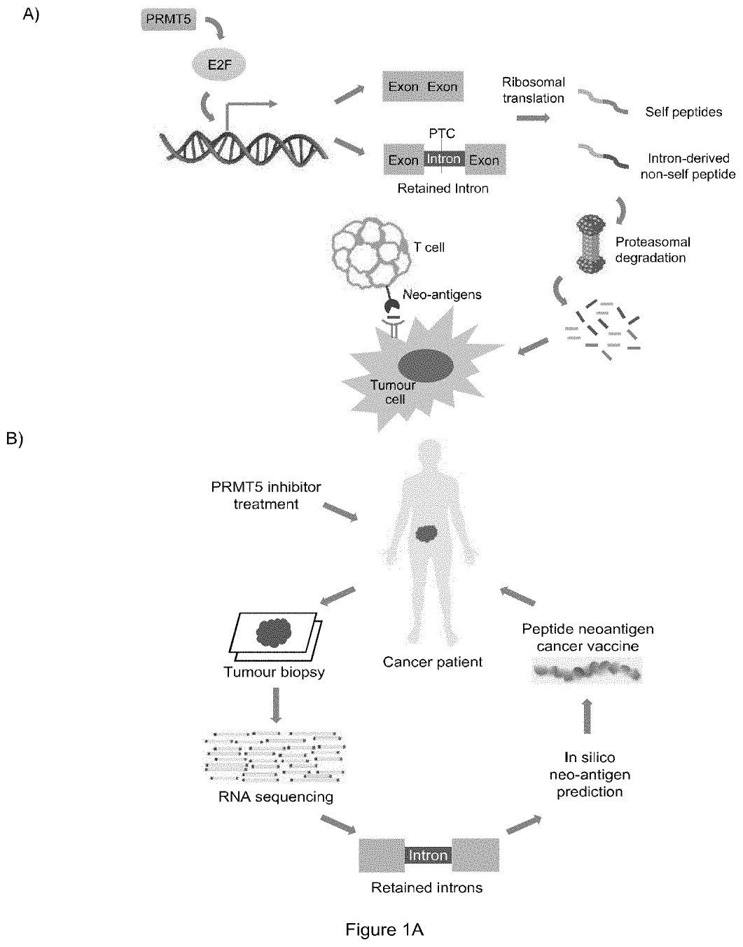

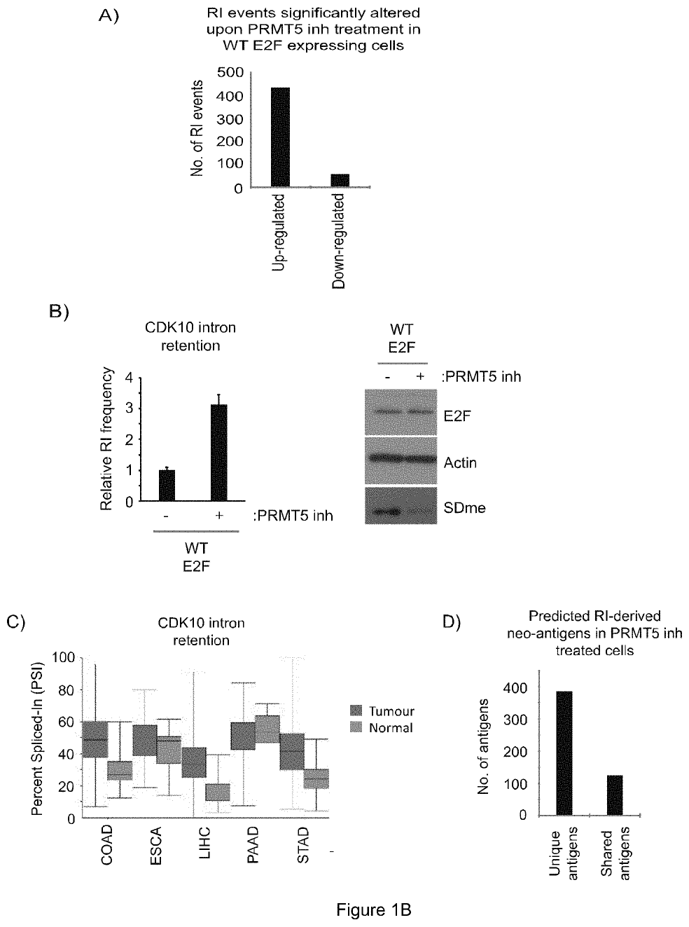

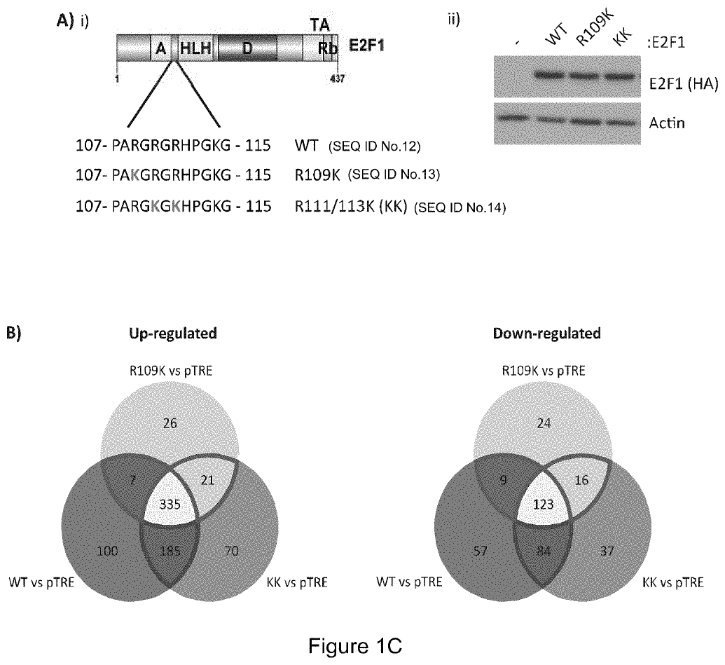

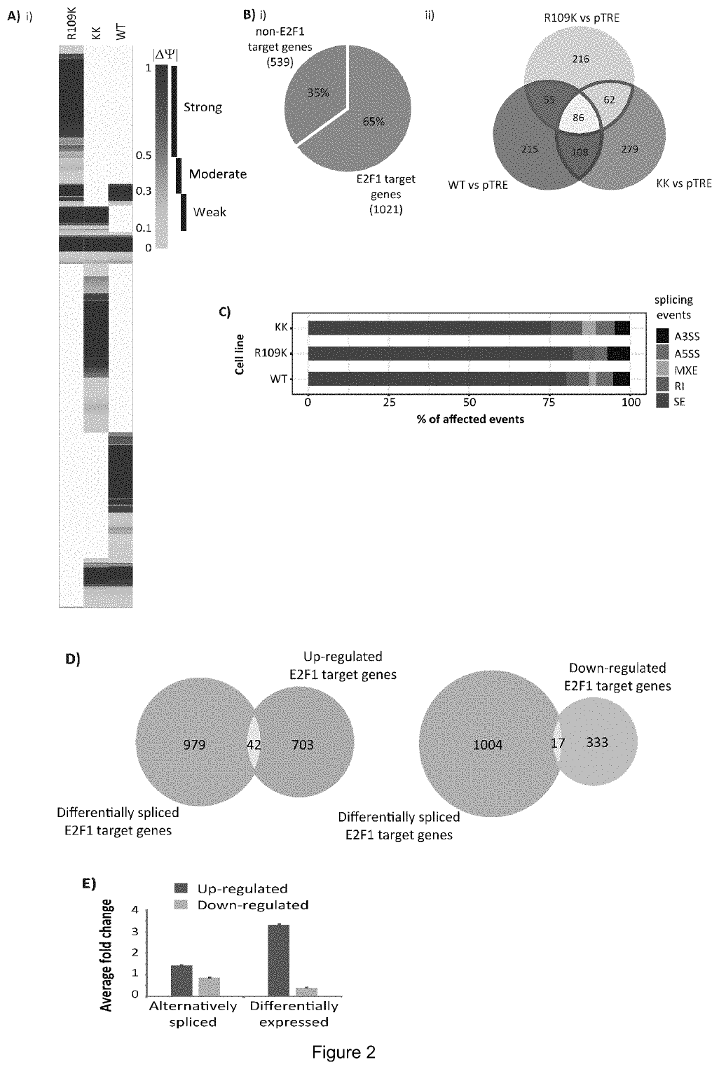

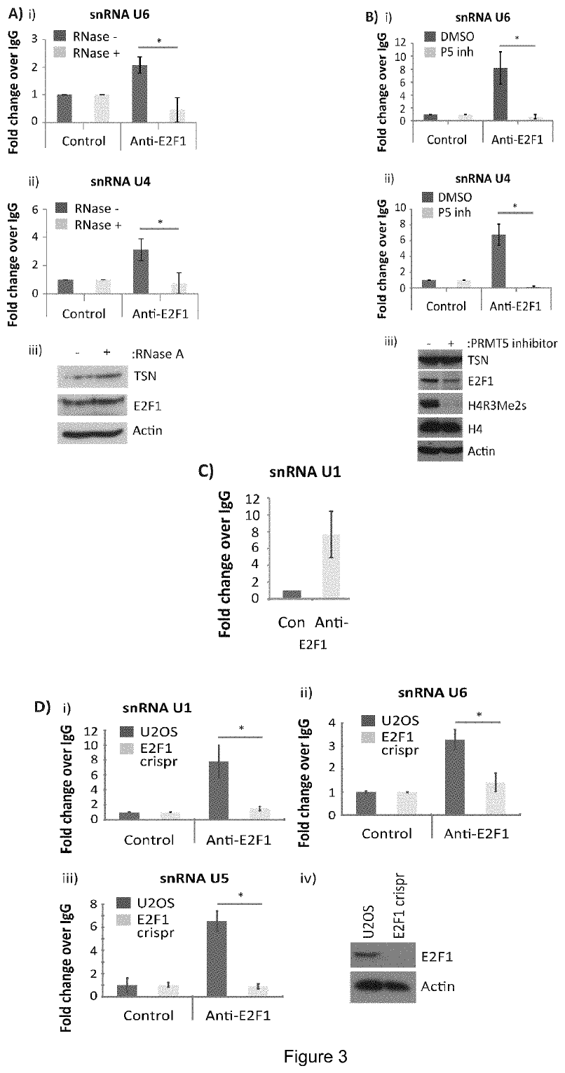

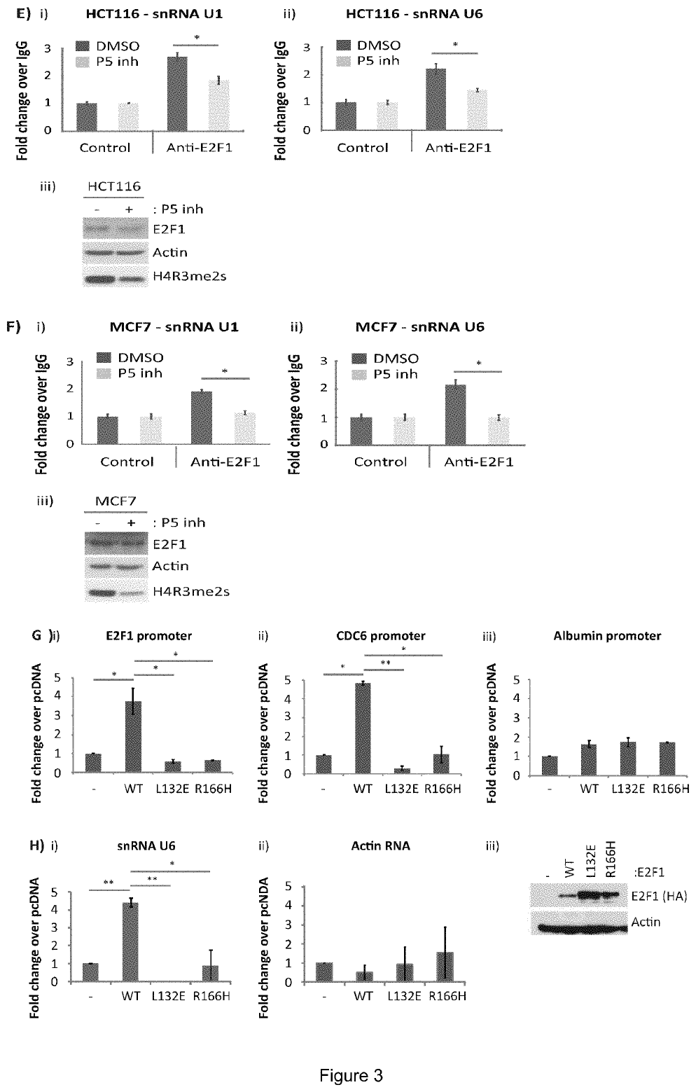

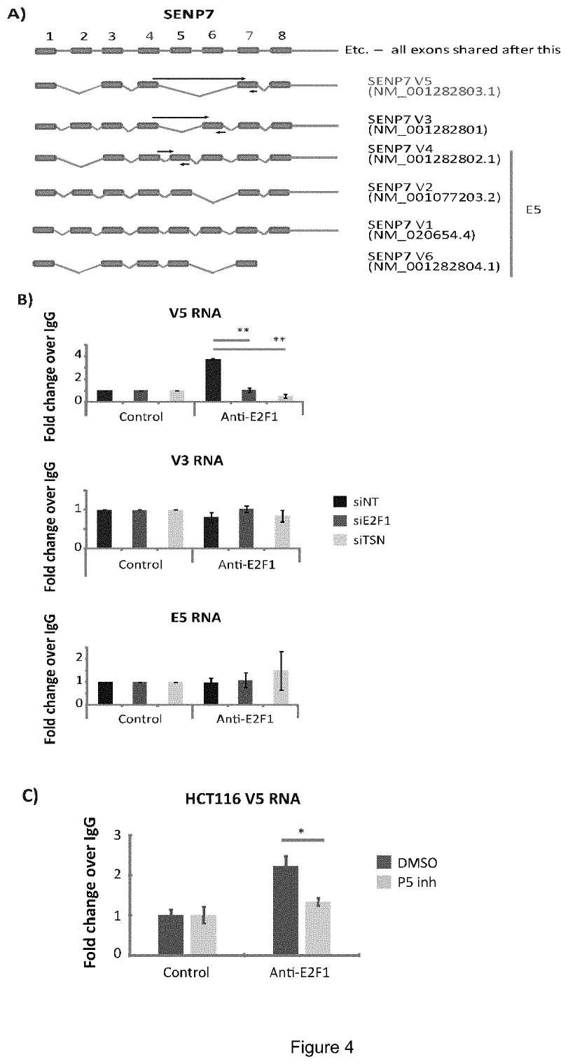

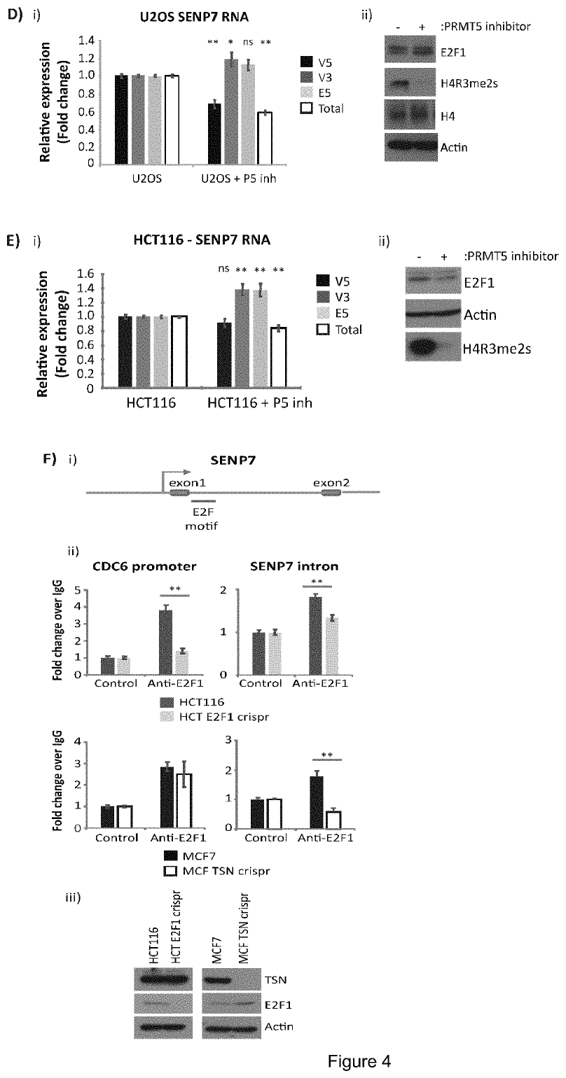

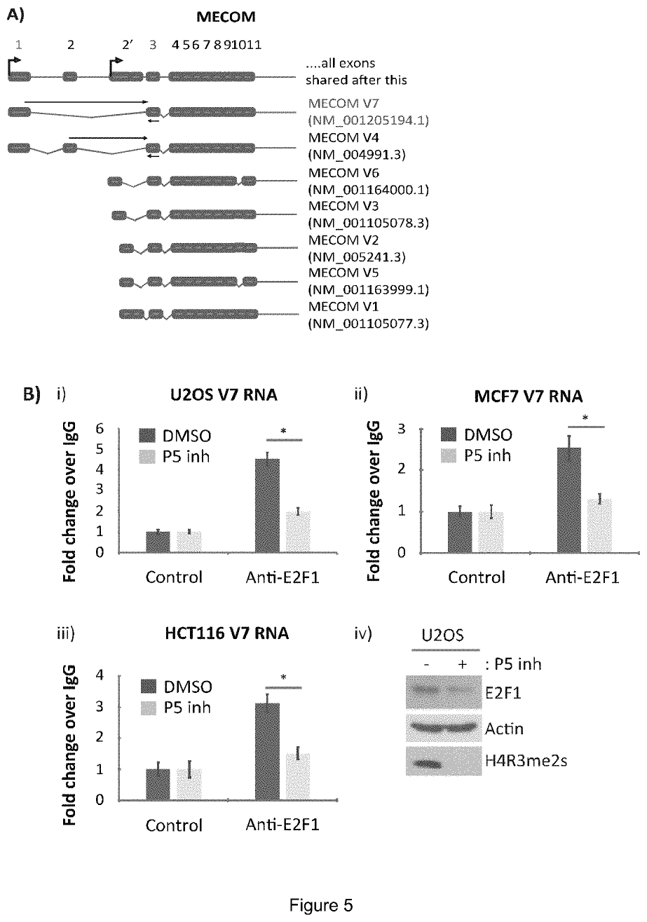

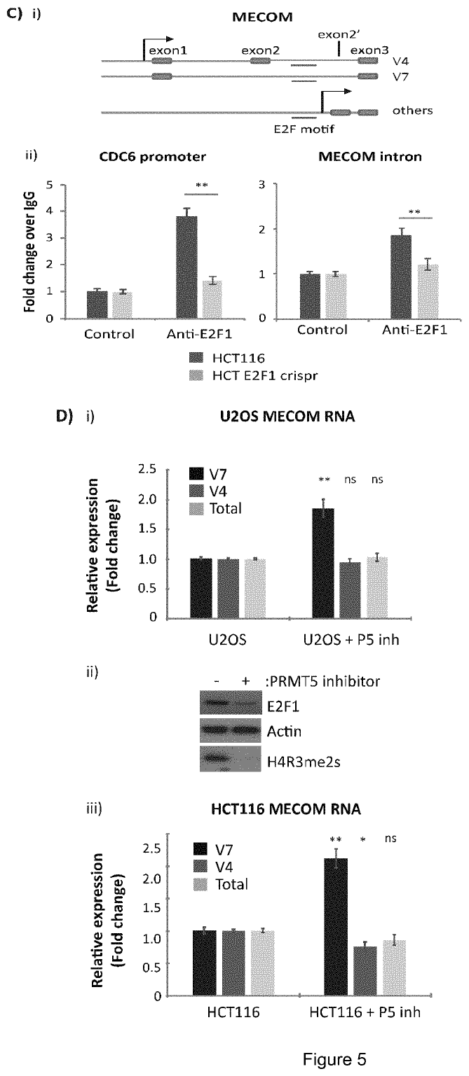

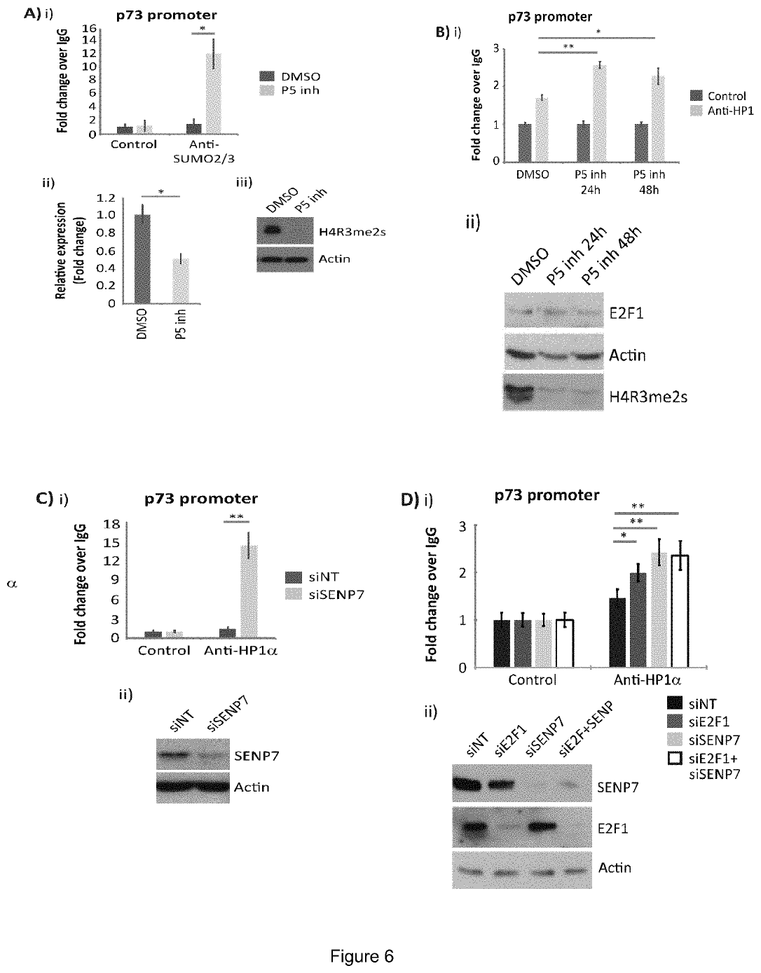

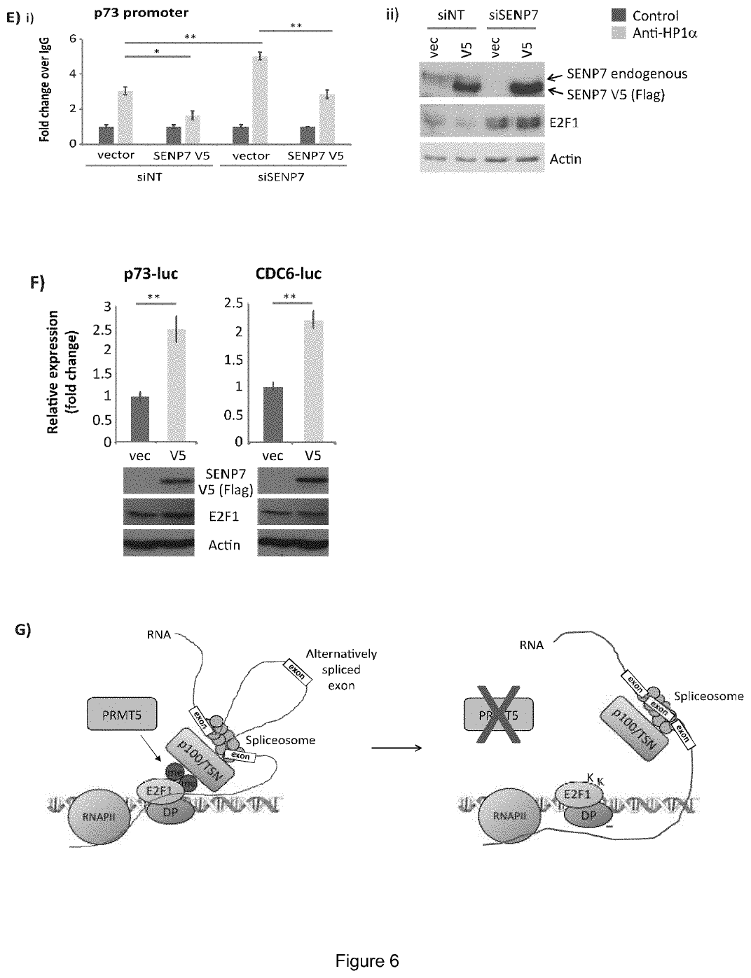

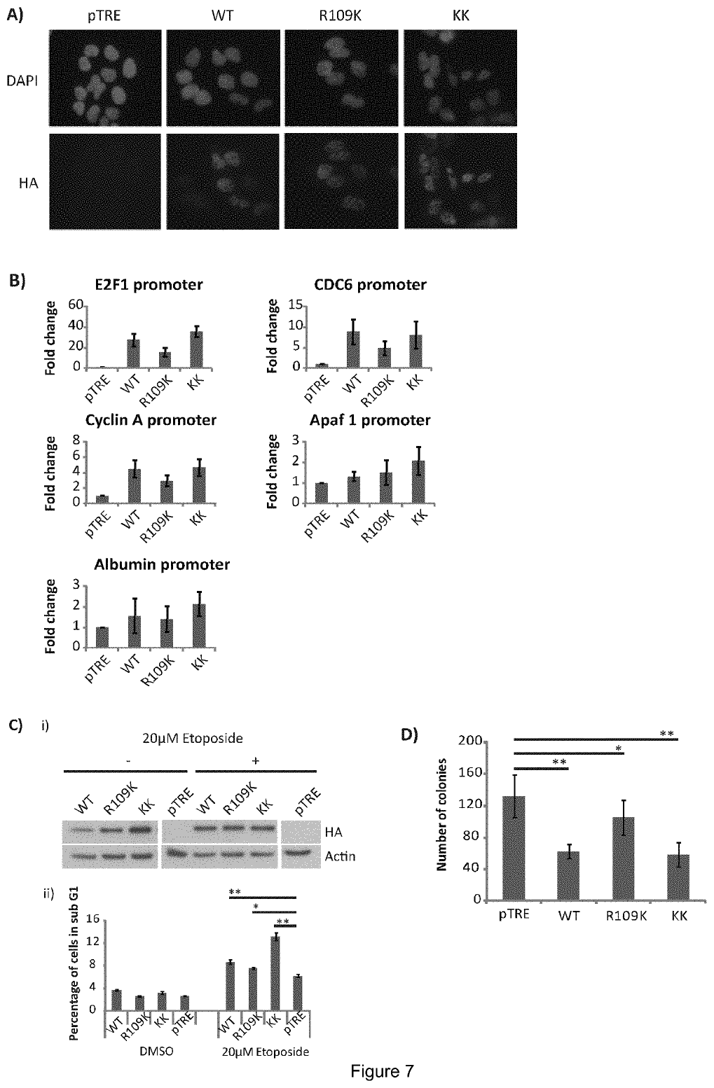

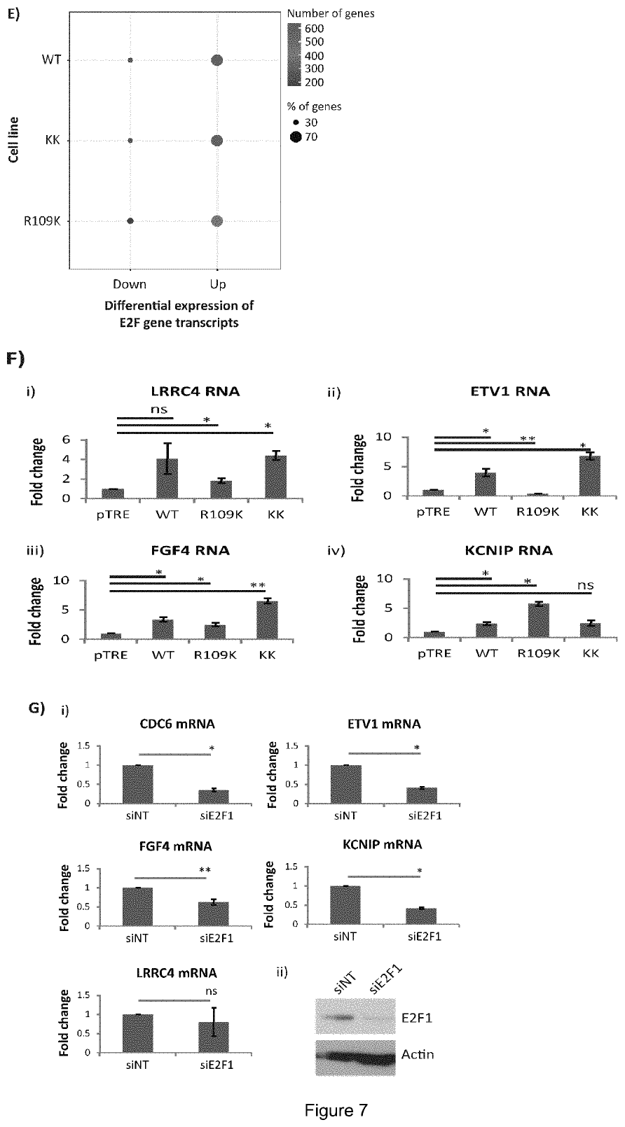

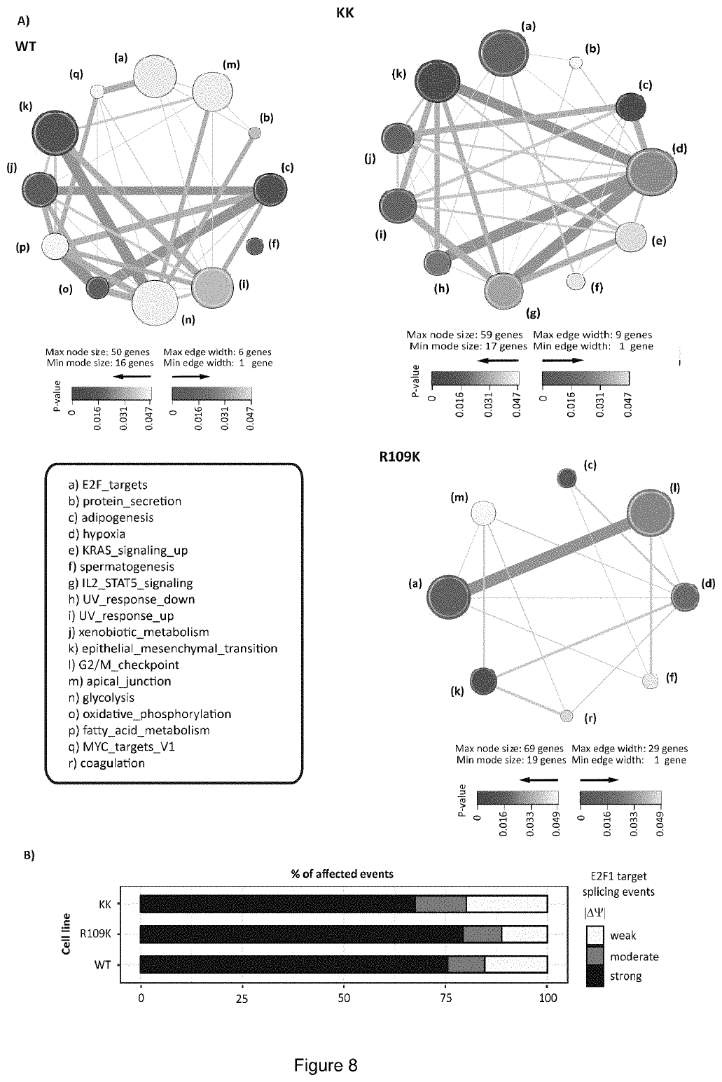

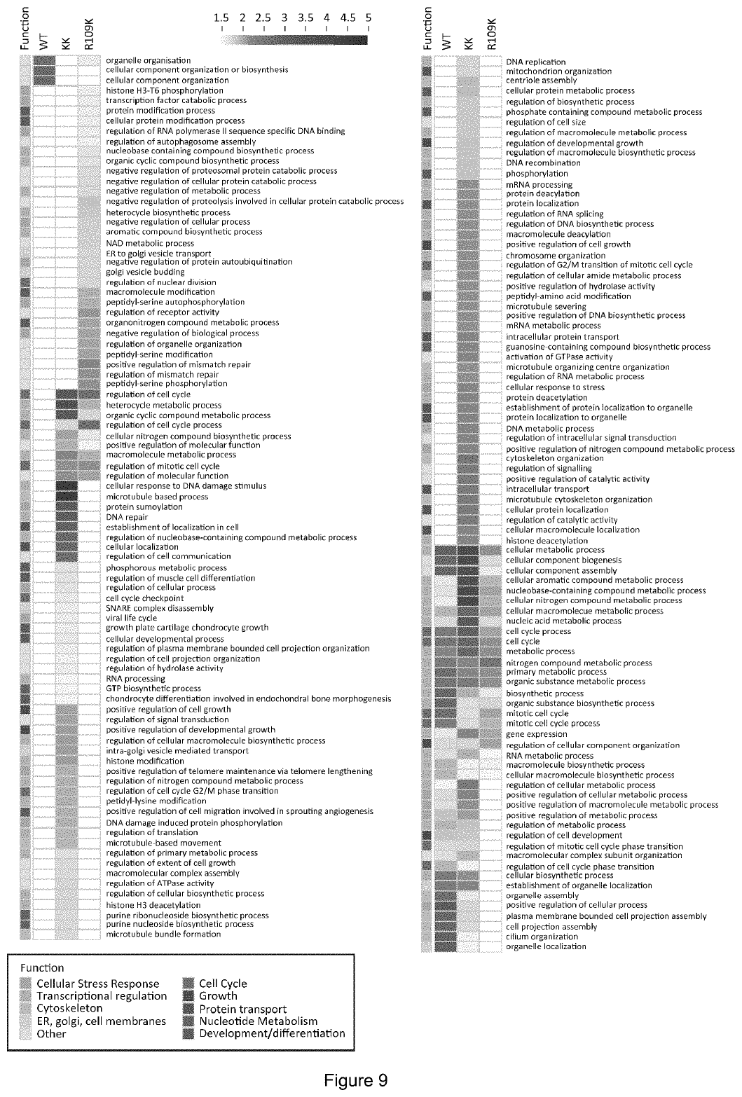

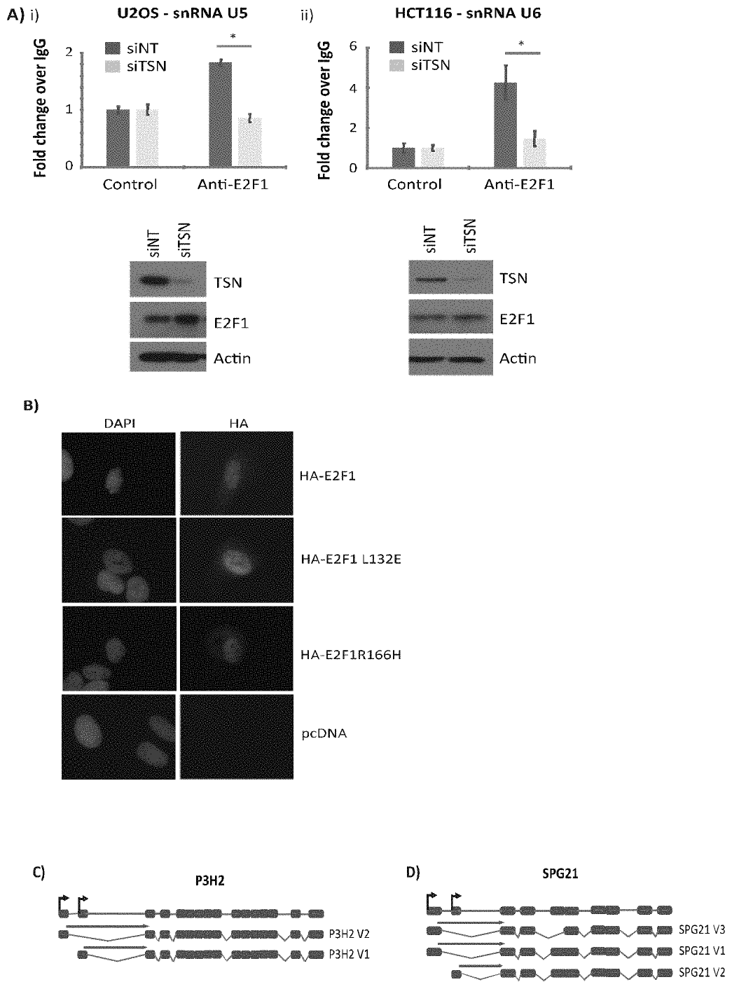

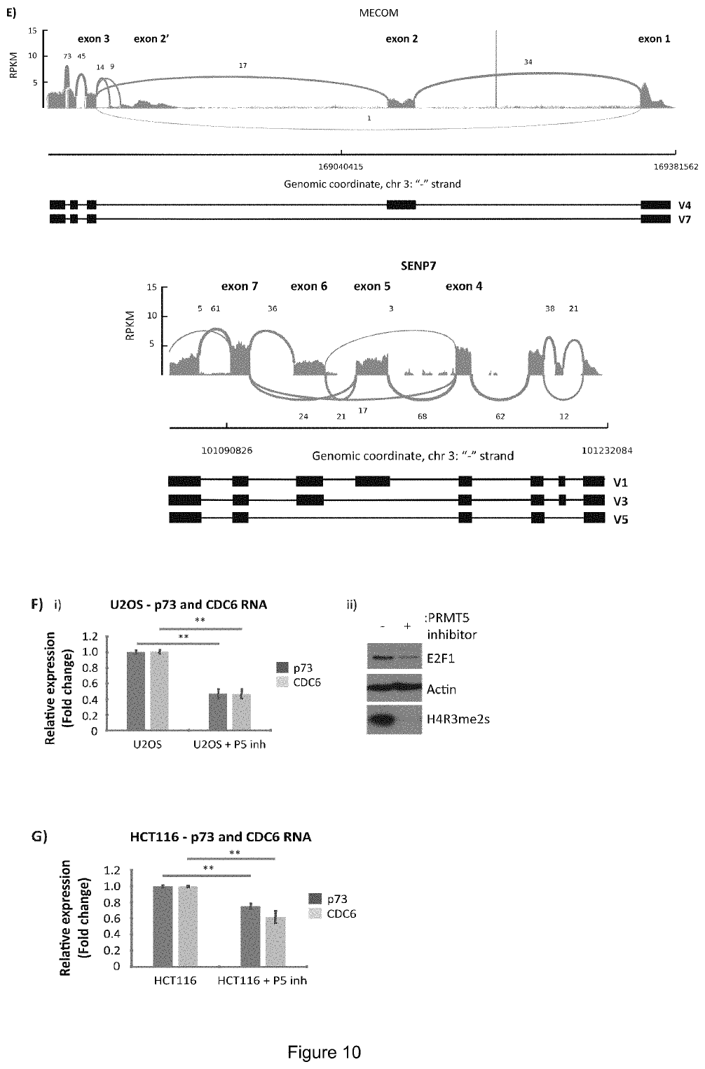

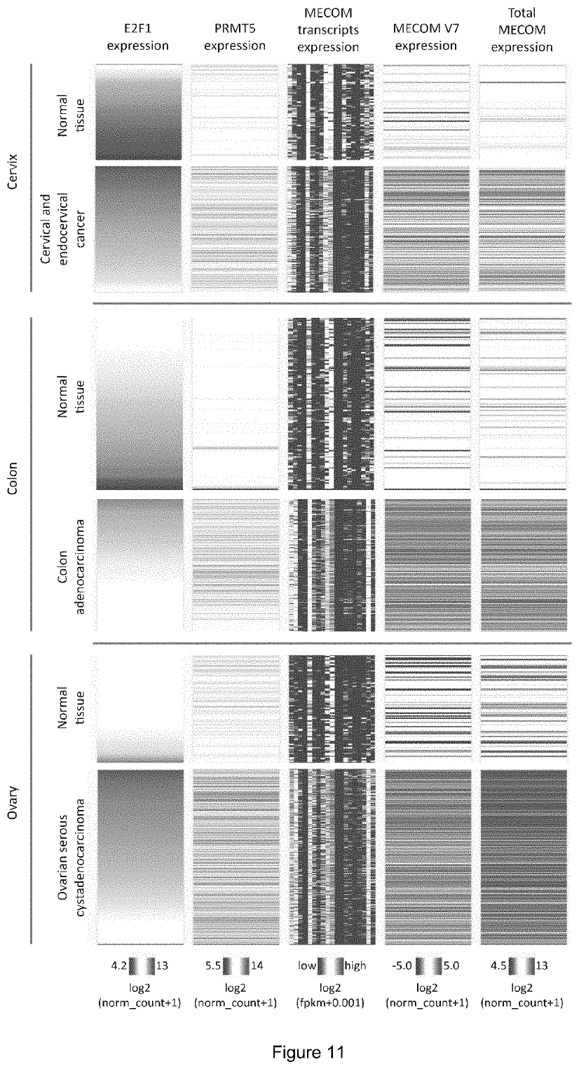

FIG. 1C: meR marks on E2F1 confer genome-wide effects.

A) Schematic representation of E2F1 highlighting the region of the protein targeted by PRMT1 and PRMT5. The arginine methylation-defective E2F1 derivatives (R109K and R111/113K [KK]) used to generate U2OS stable cell lines for RNA-seq analysis are also indicated (i). An immunoblot displaying E2F1 protein expression in U2OS stable cells after 24 h of 1 μg/ml doxycycline treatment is also included (ii). See also FIG. 7A-D.

B) Venn diagrams showing the crossover of genes up-regulated or down-regulated over 2-fold (p adj value threshold <0.01) in each cell line condition with respect to the pTRE empty vector cell line, filtered for genes containing an E2F1 motif in their proximal promoter region (−900 to +100). This data was generated from three independent biological samples.

FIG. 2: E2F1 affects alternative splicing of E2F target genes.

A) A heatmap displaying absolute values of ΔΨ (percent spliced in) for each cell line, corresponding to statistically significant alternative splicing event changes to E2F1− target genes (as determined by the presence of ChIP-seq peaks in their promoter and gene regions, retrieved from ENCODE data) with respect to the pTRE empty vector cell line, derived by analysing the RNA-seq data with rMATS algorithm. Yellow colour represents the lowest difference and blue colour represents the highest. Ivory blocks correspond to non-significant changes in splicing patterns (FDR>0.01). See also Table S2 and FIG. 9.

B) Pie chart showing the percentage of genes identified in the rMATS splicing analysis which are E2F1-target genes (as determined by the presence of ChIP-seq peaks in their promoter and gene regions, retrieved from ENCODE data) (i). The Venn diagram demonstrates the overlap of E2F1 target genes impacted by alternative splicing events (FDR<0.01) in each cell line (ii). This data was generated from three independent biological samples.

C) Bar chart displaying the statistically significant alternative splicing events to E2F-target genes for each cell line, as compared to the pTRE vector control. The percentage of these alternative splicing changes corresponding to different types of splicing event is displayed in different colours. SE=skipped/cassette exon, RI=retained intron, MXE=mutually exclusive exons, A5SS=alternative 5′ splice site, A3SS=alternative 3′ splice site. See also FIG. 8B.

D) Venn diagrams showing overlap between E2F1 target genes identified in the differential expression analysis as being up-regulated or down-regulated (regulated greater than 2-fold; FIG. 1C Panel B), and those identified as being differentially or alternatively spliced (FIG. 2A and Table S2). This data was generated from three independent biological samples.

E) Bar chart representing the average fold-change in expression of differentially expressed E2F1 target genes (regulated greater than 2-fold), compared with the expression of those E2F1 target genes where alternative splicing occurred. Only 389 genes from the alternative splicing analysis met the significance threshold for differential expression (p<0.01). The remaining 632 spliced genes had expression levels that were not significant from pTRE empty vector cell line (p>0.01), and were therefore assigned an arbitrary value of 1 for this analysis.

FIG. 3: E2F1 interacts with components of the splicing machinery.

A) U2OS cells were lysed in RIP lysis buffer, containing RNase A where indicated (20 μg/ml). Cell extracts were immunoprecipitated with E2F1 antibody and co-immunoprecipitating RNA was reverse transcribed prior to QPCR analysis with primers against U6 (i) and U4 (ii) snRNAs as indicated. Input protein levels were determined by immunoblot (iii). n=2

B) U2OS cells were treated with 5 μM PRMT5 inhibitor (P5 inh) as indicated, prior to performing an anti-E2F1 RIP. Co-immunoprecipitating U6 (i) and U4 (ii) snRNAs were identified with specific primers by qRT-PCR. Input protein levels were determined by immunoblot (iii). n=3

C) An anti-E2F1 RIP was performed on U2OS cells and co-immunoprecipitated U1 snRNA was detected by qRT-PCR. n=2

D) An anti-E2F1 RIP was performed on extracts prepared from U2OS or U2OS E2F1 CRISPR cell lines as indicated. Immunoprecipitated RNA was analysed by qRT-PCR using primers specific to U1 (i), U6 (ii), or U5 (iii) snRNA. Input protein levels are also displayed (iv). n=2

E) HCT116 cells were treated with 5 μM PRMT5 inhibitor (P5 inh) where indicated, prior to performing an anti-E2F1 RIP. Co-immunoprecipitated U1 (i) and U6 (ii) snRNA were detected by qRT-PCR. Input protein levels are also displayed (iii). n=2

F) As above, though the experiment was performed in MCF7 cells.

G) U2OS cells were transfected with 1 μg plasmid encoding wild type E2F1 (WT), DNA-binding domain mutant constructs (L132E and R166H), or empty vector (−) as indicated. 48 h later cell extracts were used for ChIP analysis with anti-HA antibody. Immunoprecipitated chromatin was analysed by QPCR using primers targeting the indicated promoters, where albumin served as the non-E2F target gene control (i to iii). Input protein levels are shown in FIG. 3H. n=2. See also FIG. 10B.

H) U2OS cells were transfected as above. 48 h later cell extracts were used for RIP analysis with anti-HA antibody. Immunoprecipitated RNA was analysed by qRT-PCR using primers specific to U6 snRNA (i) or actin RNA (ii). Input protein levels were determined by immunoblot (iii). n=3.

FIG. 4: p100/TSN enables E2F1 to interact with alternatively spliced transcripts.

A) Schematic representation of exon structure for the SENP7 gene. Each alternatively spliced transcript expressed from this gene is displayed, with primer binding sites used to detect specific transcript variants in subsequent experiments indicated with black arrows. Note that forward primers were designed to span exon junctions. Mining of the RIP-seq data set for exon spanning peaks identified reads around exons 4 and 7 (indicated by the red numbering), which occurs in SENP7 transcript V5 (highlighted in red text).

B) Anti-E2F1 RIP with U2OS cells treated with siRNA against E2F1, TSN or non-targeting control (NT) as indicated for 72 h. Cells were then immunoprecipitated with E2F1 antibody and co-immunoprecipitating RNA was reverse transcribed prior to QPCR analysis with primers against specific SENP7 transcript variants as indicated. n=3.

C) HCT116 cells were treated with 5 μM PRMT5 inhibitor (P5 inh) where indicated prior to performing an anti-E2F1 RIP. Co-immunoprecipitating SENP7 V5 transcripts were analysed by qRT-PCR. Input protein levels are the same as those displayed in FIG. 3E. n=2

D) U2OS cells were treated for 72 h with 5 μM PRMT5 inhibitor (P5 inh). RNA was then isolated from cells and analysed by qRT-PCR using primers targeting specific SENP7 transcript variants or total SENP7 RNA. Average (mean) fold change of each RNA species as compared to untreated U2OS cells was calculated and displayed with standard error. Statistical analysis for each condition compared to untreated U2OS cells is also displayed over each bar (i). An immunoblot to demonstrate input protein levels is also included (ii). n=3.

E) As above, though the experiment was performed in HCT116 cells. n=4

F) Examination of the promoter region of the SENP7 gene (−2 Kb to +1 Kb) identified an E2F1 DNA binding motif within +450 bp of the transcription start site, lying within the first intron (E2F1 motif marked in red) (i). An E2F1 ChIP was performed in HCT116 E2F1 CRISPR and MCF7 TSN CRISPR cell lines. Immunoprecipitated chromatin was analysed using primers spanning the identified E2F DNA binding motif in SENP7, or against the known E2F motif in the promoter sequence of CDC6 (ii). An immunoblot is included to demonstrate input protein levels (iii). n=3 FIG. 5: E2F1 also interacts with alternatively spliced transcripts from the MECOM gene.

A) Schematic representation of exon structure for the MECOM gene. Each alternatively spliced transcript expressed from this gene is displayed, with primer binding sites used to detect specific transcript variants in subsequent experiments indicated with black arrows. Note that forward primers were designed to span exon junctions. Mining of the RIP-seq data set for exon spanning peaks identified reads spanning exons 1 and 3 (indicated by the red numbering), which occurs in MECOM transcript V7 (highlighted in red text).

B) U2OS (i), MCF7 (ii), or HCT116 cells (iii) were treated with 5 μM PRMT5 inhibitor (P5 inh) as indicated. An anti-E2F1 RIP was then performed and co-immunoprecipitated MECOM transcript variant V7 was analysed by qRT-PCR using specific primers. Input protein levels for the U2OS experiment are also included (iv), whilst the input protein levels for HCT116 and MCF7 cells are the same as those displayed in FIGS. 3E and 3F respectively. n=2

C) Examination of the promoter region of the MECOM gene identified an E2F1 DNA binding motif lying within the first intron of V7, or the second intron of V4 (E2F1 motif marked in red) (i). An E2F1 ChIP was performed in HCT116 or HCT116 E2F1 CRISPR cell lines. Immunoprecipitated chromatin was analysed using primers spanning the identified E2F DNA binding motif in MECOM, or against the known E2F motif in the promoter sequence of CDC6 (ii). Input protein levels are the same as those displayed in FIG. 4F. n=3

D) U2OS cells (i) or HCT116 cells (iii) were treated with 5 μM PRMT5 inhibitor (P5 inh) where indicated. RNA was then isolated from cells and analysed by qRT-PCR using primers targeting specific MECOM transcript variants or total MECOM RNA. Average (mean) fold change of each RNA species as compared to untreated U2OS/HCT116 cells was calculated and displayed with standard error. Statistical analysis for each condition compared to untreated cells is also displayed over each bar. Input protein levels for U2OS cells are also displayed (ii), whilst the input protein levels for HCT116 cells are the same as those displayed in FIG. 4E. n=4

FIG. 6: Biological consequence of SENP7 alternative splicing for E2F1 activity.

A) U2OS cells were treated with 5 μM PRMT5 inhibitor (P5 inh) for 72 h where indicated, prior to ChIP analysis with anti-SUMO2/3 specific or control antibodies. Immunoprecipitated chromatin was analysed using primers specific for the E2F site in the p73 promoter (i). An RT-PCR was also performed to monitor the levels of p73 transcripts in the cell (ii). An immunoblot for H4R3me2s is included to demonstrate activity of the PRMT5 inhibitor (iii). n=3. See also FIGS. 10F and 10G.

B) As above, though cells were treated with the PRMT5 inhibitor (P5 inh) for 24 h or 48 h as indicated. ChIP analysis was performed with anti-HP1a specific or control antibodies (i). An immunoblot for H4R3me2s is included to demonstrate activity of the PRMT5 inhibitor (ii). n=2

C) U2OS cells were transfected with SENP7 siRNA or non-targeting siRNA (siNT) for 96 h as indicated. Cells were then prepared for ChIP analysis as above (i). An immunoblot is included to demonstrate input protein levels (ii). n=4

D) ChIP analysis as above, though U2OS cells were transfected with siRNA targeting E2F1, SENP7, or a combination of the two (siE2F1+siSENP7). n=3

E) U2OS cells were transfected with siRNA targeting SENP7 or non-targeting siRNA (siNT) for 96 h as indicated. Cells were subsequently transfected for 48 h with empty vector or a plasmid expressing Flag-tagged SENP7 V5. Cells were then prepared for ChIP analysis as described above (i). An immunoblot is included to demonstrate input protein levels (ii). n=3

F) U2OS cells were transfected with p73-luciferase or CDC6-luciferase reporter plasmids for 48 h, along with empty vector (vec) or Flag-tagged SENP7 V5. Reporter activity was measured and immunoblots performed to monitor input protein levels. n=2

G) Model diagram where PRMT5-mediated methylation of chromatin-associated E2F1 mediates its interaction with p100/TSN, which permits the E2F1 complex to associate with a subset of RNAs, some being derived from E2F-target genes. By regulating the activity of the splicing machinery, it is proposed that the E2F1-p100/TSN complex can influence the alternative splicing of these RNAs. In the absence of E2F1 methylation (either under conditions of PRMT5 inhibitor treatment or in cells expressing E2F1-meR point mutants), a p100/TSN-dependent interaction with the splicing machinery is lost, and changes to alternative splicing of a subset of RNAs result.

FIG. 7: Generation of stable, inducible cell lines expressing E2F1 methylation site mutants.

A) U2OS stable cell lines were treated with 1 μg/ml doxycycline for 24 h to induce expression of wild-type E2F1 (WT), E2F1 R109K, or E2F1 R111/R113K (KK) as indicated. An empty vector cell line was included as a control (pTRE). E2F1 expression and localisation was detected with an anti-HA antibody and nuclei were stained with DAPI.

B) U2OS stable cell lines were treated as above to induce expression of WT E2F1, R109K, or KK. An immunoprecipitation was performed using anti-HA antibody, and isolated chromatin was analysed by QPCR using primers targeting the indicated promoters. n=3

C) U2OS stable cell lines were treated with doxycycline as described above, then subsequently exposed to etoposide for 48 h as indicated. An immunoblot was performed to demonstrate E2F1 expression (i). Cells were also prepared for flow cytometry analysis, and the average percentage of cells in sub-G1 is displayed with standard error shown (ii). n=3

D) 1000 cells of each U2OS stable cell line was plated and treated with doxycycline to induce E2F1 protein expression for 10 days. A colony formation assay was performed and the average number of colonies per well is displayed with standard error. n=3

E) Proportion of E2F1 target genes (containing E2F1 binding motifs in their proximal promoters [−900 to +100]) from the RNA-seq analysis on each cell line that were up- or down-regulated over 2-fold (p adj value <0.01). The size of the dot reflects a percentage of the genes and the colour corresponds to the raw numbers, as indicated in the figure. See also Table S1 and FIG. 8A.

F) Wild-type E2F1 (WT), R109K or KK protein expression was induced in the U2OS stable cell lines by addition of 1 μg/ml doxycycline for 24 h. RNA was then isolated from cells and analysed by qRT-PCR using primers against target genes selected from the RNA-seq (i to iv). n=3.

G) U2OS cells were transfected with siRNA against E2F1 or non-targeting siRNA (NT) for 72 h. RNA was then isolated and qRT-PCR was performed with primers targeting the indicated genes (i). An immunoblot is also included to show input protein levels (ii). n=2

FIG. 8: Additional analysis of RNA-seq and rMATS datasets.

A) Lists of upregulated and downregulated genes from each cell line (wild type [WT], R109K, R111/113K [KK]) used in the RNA-seq analysis (FIG. 1C Panel B) were utilised for GSA analysis using the piano package. Enriched sets are represented as nodes and are coloured by significance (red indicating up-regulation and blue indicating down-regulation). Nodes are connected based on the genes they share by grey lines, with edge thickness correlating with the number of shared genes. Only differentially expressed, known E2F1 target genes with adjusted P-value <0.01 were used in the GSA analysis.

B) Magnitude of splicing changes observed in each cell line from the RNA-seq for different ΔΨ ranges: strong (>50%), moderate (30-50%), weak (10-30%), expressed as a percentage of the total events for each cell line. This bar chart is an alternative representation of the data in FIG. 2C.

FIG. 9: GO biological process enrichment analysis on spliced E2F1-target genes from the RNA-seq data GO biological process enrichment analysis was performed on the unique genes in each cell line associated with statistically significant, non-overlapping alternative splicing events (From FIG. 2A). The heatmap was generated using automatic clustering of GO:BP terms, which were then manually assigned to broader functional groupings as depicted to the bottom of the heatmap (Function).

FIG. 10: Additional analysis of E2F1 RIP-seq data sets.

A) U2OS cells (i) and HCT116 cells (ii) were transfected for 96 h with 25 nM non-targeting (siNT) or p100/TSN specific siRNA, prior to lysis in RIP buffer. Cell extracts were immunoprecipitated with E2F1 antibody and co-immunoprecipitating RNA was reverse transcribed prior to QPCR analysis with primers against U5 (i) and U6 (ii) snRNAs as indicated. Input protein levels were determined by immunoblot.

B) U2OS cells were transfected with HA-tagged wild-type E2F1, E2F1 L132E, E2F1 R166H, or empty vector (pcDNA) for 48 h as indicated. Expression and localisation of E2F1 was detected by indirect immunofluorescence using an anti-HA antibody, whilst nuclei were stained with DAPI.

C) and D) Schematic diagrams representing the exon structure for each of the indicated genes: P3H2 (C), and SPG21 (D). All annotated alternative transcripts expressed from each gene are also displayed, with transcription initiation sites highlighted by black arrows. Mining of the RIP-seq data for peaks which span exon boundaries identified a number of reads that permitted specific transcript variants to be identified. These exon spanning reads are indicated on the diagrams with red arrows.

E) Sashimi plots of RNA-seq data for the MECOM and SENP7 gene are displayed to demonstrate that transcript variants observed in the RIP-seq (FIGS. 4 and 5) could also be observed in the RNA-seq data (FIG. 2). The genomic coordinates for the highlighted regions of the gene, and a schematic of the splicing events occurring, are shown at the bottom of each graph. Exons are labelled as per the numbering system used in FIG. 4A (SENP7) and FIG. 5A (MECOM). The main panel shows the count of RNA-seq reads that span the exon junctions in this region of the gene (taken from the WT E2F1 expressing cells).

F) U2OS cells were treated with 5 μM PRMT5 inhibitor (P5 inh) as indicated, prior to RNA extraction and qRT-PCR analysis with primers recognising total p73 or CDC6 transcripts as indicated (i). Input protein levels are also displayed (ii). n=4

G) As above, though the experiment was performed in HCT116 cells. Input protein levels are the same as those displayed in FIG. 4E. n=3

FIG. 11: Expression of E2F1, PRMT5 and MECOM V7 RNA variant in human cancer.

Heatmap representation of expression levels for E2F1, PRMT5, and MECOM V7 transcripts in different cancers (cervical, colon and ovarian cancer) compared to normal tissue, generated using Xena Browser. Data from The Cancer Genome Atlas and Genotype-Tissue Expression projects were used to display expression levels from cancer tissue or healthy tissue respectively. FPKM; Fragments Per Kilobase of transcript per Million mapped reads.

### SUPPLEMENTARY DATA (NOT SHOWN)

Substantial additional data were generated but not shown as follows.

Table S1: List of upregulated and downregulated E2F1 target genes identified from the RNA-seq analysis for each cell line, corresponding to FIG. 1C Panel B.

Table S2: List of alternative splicing events in E2F1 target genes identified in the RNA-seq rMATS analysis corresponding to the heatmap (FIG. 2A).

Table S3: Differential expression of genes associated with RNA splicing, taken from the RNA-seq dataset (FIG. 1C Panel B).

Table S4: List of RNA sequences identified in the anti-E2F1 RIP-seq analysis (FIG. 4).

Table S5: List of over-lapping E2F target genes between RIP-seq dataset (FIG. 4) and splicing analysis (FIG. 2A).

Table S6: List of E2F1 RIP-seq reads that span exon junctions.

### DETAILED DESCRIPTION OF THE INVENTION

The present inventors have highlighted RNA splicing as a source of neo-antigens, that can be manipulated to therapeutic benefit.

Alternative splicing (AS) permits the expression of multiple protein isoforms from a single gene, and it has been established that splicing is relevant to human disease, including cancer, where defects in the splicing machinery can give rise to tumour-specific splicing alterations and the expression of cancer-specific protein isoforms frequently with novel biological functions (El Marabti E. et al. 2018). Splicing involves the removal of introns from pre-mRNA, which are usually short-lived, to produce mature translatable mRNA. Intron retention, though very rare in normal cells, arises from a failure to remove an intron from mature mRNA. Retention of intronic sequences usually results in the introduction of a premature termination codon, which when translated target the mRNA for destruction via the nonsense-mediated decay pathway (NMD) (Smart A. C. et al. 2018). However, truncated, non-functional proteins are generated, which contain foreign protein sequence derived from translating the intronic RNA sequence (FIG. 1A). The invention is based in part on the finding that these intron-derived foreign sequences can generate non-self peptides, which upon endogenous processing via the proteasome act as high affinity peptides, namely neo-antigens, for presentation to the immune system by HLA molecules. The ability to regulate the presence of stable RI containing RNAs provides us with a powerful approach to therapeutically manipulate the repertoire of neo-antigens.

The invention arose from detailed cancer biology studies on the E2F pathway where methylation of arginine residues (meR) is a central mechanism for channelling E2F through its distinct biological pathways. The meR mark, supplied by the methyl-transferase PRMT5, integrates E2F's normal transcriptional regulatory role with the splicing machinery, enabling gene transcription and RNA splicing to be co-ordinated. Treating cancer cells with a PRMT5 inhibitor, which blocks the meR mark on E2F, produces a new set of stable RI containing RNAs (FIG. 1B).

It has been shown in the Examples that methylation by PRMT5 enables E2F1 to regulate a diverse group of genes at the level of alternative RNA splicing.

Without wishing to be bound by theory, methylated E2F1 recruits spliceosome machinery and thereby enables the splicing out of introns from pre-mRNA transcripts. Accordingly, inhibiting methylation (e.g. by PRMT5 inhibition) of E2F1, reduces recruitment to the spliceosome, reduces splicing, and leads to (increased) retention of introns. These retained introns in RNA transcripts may therefore be expressed in proteins, as neoantigens.

Thus, the inventors have provided an approach where, by manipulating arginine methylation, it is possible to regulate the production of stable RIs in cancer cells. Small non-functional protein sequences derived from translating stable RI containing RNAs are expected to produce a new repertoire of neo-antigens which cancer cells present to the immune system, providing a new and unprecedented opportunity for developing cancer vaccines with very wide clinical utility.

This is a previously unexplored approach towards regulating the neo-antigen landscape of tumour cells. The strategy has arisen from bringing together genome-wide studies on arginine methylation, E2F pathway control and RNA splicing, allowing the inventors surprisingly to identify a new way to create a stable, deregulated splicing programme in cancer cells, causally dependent on arginine methylation and E2F activity, where a diverse set of RNAs with a retained intron are stably expressed. Without wishing to be bound by theory, the inventors assert that this is an important mechanism for regulating the neo-antigen landscape which can be exploited therapeutically to develop cancer vaccines to augment the adaptive immune response. Many cancer drugs have limited applications since they target shared pathways between normal and tumour cells, and hence often have adverse effects. Because a neoantigen derived cancer vaccine would selectively target tumour cells, it offers a powerful therapeutic option for reducing side effects in patients.

The potential of personalised vaccines for cancer immunotherapy was recently described by Sahin and Tureci (Science, 23 Mar. 2018: Vol 359, Issue 6382, pp 1355-1360). This paper observes that technological advances in genomics, data science, and cancer immunotherapy now enable the on-demand production of a vaccine (e.g. neoantigen) therapy customised to a patient's individual tumor. This paper also notes that one of the critical challenges for personalised cancer vaccines is accurately to map the cancer mutanome, so as to select the most suitable mutations for optimal immune responses. The invention described herein provides a powerful tool for identifying and regulating the neoepitopes in a cancer.

A key attribute of the invention is the potential for very wide clinical utility; many cancers, for example oesophageal, pancreatic, gastric and hepatic cancers, are poorly responsive to standard therapies including immune checkpoint approaches. The RI neo-antigen approach described herein can, in certain embodiments, be used to treat these difficult-to-treat cancers.

The ability to manipulate the neoantigen landscape of cancer cells assists significantly in one of the key clinical challenges in this area, namely the identification of neoantigens and cognate T cells from a cancer patient in need of therapy.

### Retained Introns Contribute to the Neo-Antigen Landscape of Cancer Cells

It has been found that retained introns contribute to the neo-antigen landscape of cancer cells, and that it is possible to regulate expression of neoantigens resulting from intron retention. This is conveniently summarised in panel A of FIG. 1A. In particular, retained introns in E2F-target genes have been shown to contribute to the neo-antigen landscape of cancer cells. Arginine methylation of E2F by PRMT5 connects its transcriptional regulation activity with that of alternative splicing (AS) regulation.

E2F-target gene transcript variant choice is therefore determined in part by the methylation status of E2F in cells. In particular, cells treated with PRMT5 inhibitors (wherein E2F methylation is decreased) demonstrate an unexpectedly large proportion of RNAs containing stably retained introns (RI). These introduce premature termination codons (PTC) that give rise to truncated, non-functional proteins containing foreign (non-self) amino acid sequences derived from the intronic sequence (shown in red in FIG. 1A, panel A).

When these truncated proteins are processed by the endogenous cellular machinery for presentation on HLA (MHC) molecules at the cell surface, they give rise to non-self peptides which have the potential to be recognised by the patient's adaptive immune system. In contrast, peptides derived from wild-type proteins are recognised as self and would not elicit an immune response. Use of PRMT5 inhibitors to regulate the presence of RI-containing RNAs therefore provides a powerful approach to therapeutically manipulate the neo-antigen landscape of cancer cells.

Patients with cancers, including cancers that are difficult to treat with conventional drugs or immunotherapies, may in some embodiments benefit from a personalised neo-antigen cancer vaccine approach. This general approach is depicted in FIG. 1A panel B, and in one embodiment the invention comprises a method involving the steps set out in that Figure. Cancer cells, typically tumour tissue, from a patient can be sampled (by biopsy or any other suitable technique to obtain RNA transcripts from the cancer) and RNA-sequencing can be performed to identify the population of RNAs containing RIs. Optionally, the patient is treated with an E2F inhibitor, optionally a PRMT5 inhibitor, prior to sampling the tumour to regulate the presence of RI-containing RNAs. RNAs from a population of patients may also be assessed to determine RI-containing RNAs that may be present in a particular cancer type.

RI-derived peptides can then be predicted based on the RNA data, which peptides could act as high affinity neo-antigens for presentation on HLA class I molecules. In one embodiment, an in silico neo-antigen algorithm can be used to predict which peptides could act as high affinity neo-antigens. Suitable neoantigen prediction algorithms are known in the art, for example as described by Yadav M. et al. 2014; Cohen C. J. et al. 2015; Bassani-Sternberg M. et al. 2016; and Freudenmann L. K. et al. 2018.

Neoepitope candidates can be selected to design peptide vaccines that can then be administered to the patient to improve the immune recognition of cancer cells and promote tumour regression.

The term “neoantigen” or “neoepitope” refers to antigens that are not encoded in a normal, non-mutated host genome. A neoantigen refers to an antigen including one or more amino acid modifications compared to the parental antigen. For example, a neoantigen may be a tumor-associated neoantigen, wherein the term “tumor-associated neoantigen” can include a peptide or protein including amino acid modifications due to tumor-specific mutations. For example, a neoantigen may be a disease-associated neoantigen, wherein the term “disease-associated neoantigen” can include a peptide or protein including amino acid modifications due to disease-specific mutations. In some instances, a neoantigen represents either oncogenic viral proteins or abnormal proteins that arise as a consequence of somatic mutations. For example, a neoantigen can arise by the disruption of cellular mechanisms through the activity of viral proteins. Another example can be an exposure of a carcinogenic compound, which in some cases can lead to a somatic mutation. This somatic mutation can ultimately lead to the formation of a tumor/cancer.

The term “immunogenic peptide” or “immunogenic epitope” refers to an antigen, such as a neoantigen, that modulates T cells or elicits an immune response when administered to a subject. For example, an immunogenic peptide can be comprised within a vaccine. For example, an immunogenic peptide can be a peptide that activates T cells. For example, an immunogenic peptide can be a peptide that stimulates T cell proliferation. For example, an immunogenic peptide can be a peptide that stimulates T cell proliferation ex vivo. For example, an immunogenic peptide can be a peptide that is bound to an MHC of an antigen-presenting cell (APC). For example, an immunogenic peptide can be a peptide that is bound to an MHC of an APC of an APC:T cell conjugate. For example, an immunogenic peptide can be a peptide loaded onto a dendritic cell (DC) that stimulates or activates CD4+ and/or CD8+ T-cell proliferation.

### Neoepitope-Modifying Agents

The invention relates, in part, to the identification and use of agents that are able to modify or modulate the expression of neoantigens by a cell, typically a cancer cell. Whether or not an agent is able to modify, regulate or modulate neoantigen expression by a cell can be tested using assays known in the art. In certain embodiments, the agent increases the number of retained introns that are expressed by the cell.

In the Examples below, a cell is contacted with an agent in vitro and RNA sequencing used to assess the global transcript profile in each cell type. Transcripts that were upregulated 2-fold or more were selected in that Example, but an increase of 3-fold, four-fold or more could be used. In other embodiments, an increase of 20%, 50%, 75%, 80%, 90%, 100%, 200%, 300% or more could be used. It is envisioned that up-regulation will typically be used, but down-regulation could be used in some embodiments.

Within a population of differentially-regulated (e.g. up-regulated) transcripts following contact with the agent, the unique transcripts can be identified. The gene sets present in the RNA-seq may be analysed by Gene Set Analysis (GSA).

Alternative splicing within the gene transcripts can be identified using a known algorithm, for example the rMATS algorithm described by Shen et al (reference 17 below) and explained in the section “RNA-seq data analysis” in the Examples. In particular, an increase in retained introns is typically identified in the cells following contact with the agent. Measurement of ΔΨ can be used to determine whether there is an increase in alternatively spliced transcripts, such as retained introns. Typically, the FDR threshold for differential PSI can be taken as 0.01.

In one embodiment, an increase in intron-retention is determined by a simple increase in the RI events that are observed following incubation with the cell. In some embodiments, an increase of at least 50 RI events is observed, or at least 100 RI events, or at least 200 RI events, for example 300 RI events or more. This is as shown, for example, in panel A of FIG. 1B. This may also be assessed as a percentage, for example an increase in RI events of at least 20%, at least 30% m at least 40%, at least 50%, or at least 100%.

In some embodiments, intron retention is observed in CDK10. In further embodiments, intron 8 of the CDK10 gene is retained.

In some embodiments, the agent that is able to modify or modulate the expression of neoantigens by a cell, in particular the number of retained intron-containing neoantigens, is an antisense molecule, an shRNA, an siRNA, a small molecule such as a molecule with a molecular weight less than 750 Da or less than 500 Da, a protein, an antibody or an antibody fragment. As noted herein, an inhibitor of PRMT5 expression or methylation activity is a typical agent according to the invention.

### Identification of Neoepitopes

In an exemplary embodiment, RNA-seq data from a cell treated with an agent can be analysed for statistically significant (FDR<0.01) changes in alternative splicing events of genes. The number of intron retention events observed to be significantly up-regulated or down-regulated, as compared to untreated cells, can be determined. The same cell type can be treated with the agent (e.g. PRMT5 inhibitor) as per the conditions used for the RNA-seq. Total RNA can be isolated and qRT-PCR performed using primers to detect the retention of an intron of interest. In FIG. 1B, this is intron 8 of the CDK10 gene, but any intron of interest can be detected by adjusting the qRT-PCR primers and conditions. An immunoblot can be used to indicate protein levels. A blot with a control to confirm activity of the agent can also be performed, for example a blot with antisymmetric di-methyl antibody (SDme) can be used as a control to confirm activity of a PRMT5 inhibitor.

TGCA datasets from cells of interest can be examined for the presence of one or more retained introns of interest (e.g. intron 8 in the CDK10 gene in the Example shown in FIG. 1B) using TCGA SpliceSeq software (MD Anderson Cancer Center; https://bioinformatics.mdanderson.org/TCGASpliceSeq.index.jsp). The percent spliced-in values from tumour and adjacent normal samples can be calculated.

Neo-antigen prediction can then be performed using known algorithms on the RNA-seq data from wild-type cells treated with an agent of the invention (e.g. PRMT5 inhibitor) to determine potential RI-derived neoantigens that are predicted to have strong affinity for HLA. As shown in panel D of FIG. 1B, the numbers of unique and shared RI-derived antigens, as compared to untreated cells can be assessed.

### E2F and PRMT5 Activity Influences Intron Retention

The Examples show that PRMT5 inhibitors cause statistically significant (FDR<0.01) changes in alternative splicing events of E2F-target genes. This can be seen, for example, in FIG. 1B.

Accordingly, in some embodiments, the agent is an inhibitor of an E2F protein, optionally an inhibitor of PRMT5. Inhibitors of E2F and of PRMT5 are known, as described in WO-A-2011/077133, WO-A-2018/167269 and WO2018-A-167276, each of which is incorporated herein by reference in its entirety.

In the Examples below, the compound EPZ015666 from Chan-Penebre et al 2015 (incorporated herein by reference in its entirety) is used as a PRMT5 inhibitor. EPZ015666 is a potent and selective inhibitor of PRMT5 with antiproliferative effects in both in vitro and in vivo models of Mantle Cell Lymphoma. EPZ015666 is also known as GSK3235025 and has the CAS Number 1616391-65-1. As with other therapeutic agents provided by this disclosure, it may be provided as a salt, solvate or hydrate. Its structure is shown below:

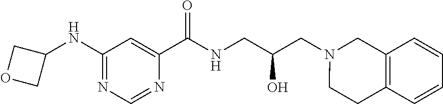 [SMILES: O=C(NC[C@H](O)CN1CCc2ccccc2C1)c1cc(NC2COC2)ncn1]

E2F is a family of transcription factors implicated in a variety of cell fates including proliferation, apoptosis and differentiation (Stevens and La Thangue; 2003; Frolov and Dyson 2004, Polager and Ginsberg 2008; van den Heuvel and Dyson 2008). E2F proteins share the capacity to regulate a diverse group of target genes (Frolov and Dyson 2004; van den Heuvel and Dyson 2008). The first family member identified, E2F-1, physically interacts with the retinoblastoma tumour suppressor protein pRb, which negatively regulates E2F-1 activity (Bandara and La Thangue 1991; Zamanian and La Thangue 1992; Weinberg 1995; Stevens and La Thangue 2003). Whilst it is established that E2F-1 can promote proliferation, it has also become clear that E2F-1 can prompt apoptosis (van den Heuvel and Dyson 2008, Polager and Ginsberg 2008). In Rb−/− mice, the enhanced levels of apoptosis in certain tissues reflect deregulated E2F-1 activity (Tsai et el 1998; Iaquinta and Lees 2007). Further, E2F-1−/− mice suffer from an increased incidence of tumours (Field et al 1996), suggesting that E2F-1 adopts a tumour suppressor role in some tissues, perhaps reflecting its ability to induce apoptosis.

In some embodiments of the invention, the agent that is able to modify or modulate the expression of neoantigens by a cell, in particular the number of retained intron-containing neoantigens, is an agent that reduces the expression and/or activity of the enzyme PRMT5. Typically the substance reduces the catalytic activity of PRMT5. In particular the substance typically reduces or abolishes the ability of PRMT5 to methylate E2F1 protein.

The sequence information for PRMT5 may be found at the National Center for Biotechnology Information (http://www.ncbi.nlm.nih.gov/pubmed/) under accession numbers NM_006109 (nucleotide) and NP_006100 (protein). The human PRMT5 protein sequence is provided below for reference:

SEQ ID No. 1:

| | |
|---|---|
| 1 | maamavggag gsrvssgrdl ncvpeiadtl gavakqgfdf |
| | lcmpvfhprf krefiqepak |
| 61 | nrpgpqtrsd lllsgrdwnt livgklspwi rpdskvekir |
| | rnseaamlqe lnfgaylglp |
| 121 | afllplnqed ntnlarvltn hihtghhssm fwmrvplvap |
| | edlrddiien aptthteeys |
| 181 | geektwmwwh nfrtlcdysk riavaleiga dlpsnhvidr |
| | wlgepikaai lptsifltnk |
| 241 | kgfpvlskmh grlifrllkk evqfiitgtn hhsekefcsy |
| | lqyleylsqn rpppnayelf |
| 301 | akgyedylqs plqplmdnle sqtyevfekd pikysqyqqa |
| | iykclldrvp eeekdtnvqv |
| 361 | lmvlgagrgp lvnaslraak qadrriklya veknpnavvt |
| | lenwqfeewg sqvtvvssdm |
| 421 | rewvapekad iivsellgsf adnelspecl dgaqhflkdd |
| | gvsipgeyts flapissskl |
| 481 | ynevracrek drdpeagfem pyvvrlhnfh qlsapqpcft |
| | fshpnrdpmi dnnryctlef |
| 541 | pvevntvlhg fagyfetvly qditlsirpe thspgmfswf |
| | pilfpikqpi tvregqticv |
| 601 | rfwrcsnskk vwyewavtap vcsaihnptg rsytigl |

By ‘reduces the expression and/or activity of the enzyme’ it is meant that expression of the enzyme is reduced or inhibited and/or that the activity of the enzyme is reduced partially or completely. Expression of the enzyme may be altered by gene therapy or by disrupting transcription of the gene encoding the enzyme or by destruction of the gene transcript or by disrupting translation or by degradation of the enzyme. The activity of the enzyme may be altered by a competitive or non-competitive inhibitor, or by genetic modification. Typically the substance reduces the catalytic activity of PRMT5. In particular the substance typically reduces or abolishes the ability of PRMT5 to methylate E2F1 protein.

Typically the method provides a reduction in the amount of active enzyme of from 10% to 100% based on the amount of active enzyme in the cell prior to treatment. Most typically the method provides a reduction in the amount of active enzyme of at least 20%, at least 30%, at least 40%, at least 50%, at least 60%, at least 70%, at least 80%, at least 90%, at least 95%, or at least 99%, based on the amount of active enzyme in the cell prior to treatment. The amount of active enzyme may be quantitated by measuring the enzyme activity in the cell or in an in vitro assay. For example, a recombinant PRMT5 enzyme may be expressed and the activity of methyltransferases measured in vitro, as described, for example, in Example 1, FIG. 1b and the Materials and Methods of WO-A-2011/077133.

Briefly, a suitable in vitro methyltransferase assay comprises a Flag-PRMT5 plasmid transfected into cells (e.g U2OS) for 48 h. Cells are lysed and Flag-PRMT5 immunoprecipitated with agarose beads and then eluted. The eluate is mixed with recombinant substrates (GST fusion proteins or histones) in methylation reaction buffer (50 mM Tris, 0.1 mM EDTA, 50 mM NaCl) with ³H labeled SAM (as —CH₃ group donor) to a volume of 40 μl and incubated at 30° C. for 90 min. Half of the reactions are then spotted onto p81 membrane circles (Whatman) and air dried. The membranes are then washed three times, 5 min each in 50 ml of wash buffer (46 mM NaHCO₃, 4 mM Na₂CO₃, pH 9.2). After washing briefly with acetone, membranes are air dried, placed in scintillation vials, immersed in scintillation fluid (Beckman Coulter) and disintegrations per minute (DPM) measured in a scintillation counter. The other half of the reactions are run on SDS-PAGE gel and used to detect ³H auto-radioisotope/methylation signal.

In one embodiment, the agent is capable of antisense inhibition or RNA interference (RNAi). The antisense molecule, shRNA or siRNA, can be for example an oligonucleotide comprising the sequence:
5′ CCGCUAUUGCACCUUGGAA (SEQ ID No.2), or a sequence with at least 90% identity thereto, or
5′ CAACAGAGAUCCUAUGAUU (SEQ ID No.3), or a sequence with at least 90% identity thereto.

Such oligonucleotides may be modified to improve their stability and/or potency and/or may be modified to enable systemic delivery.

In another embodiment the agent is a small molecule inhibitor of PRMT5. This may be less than 750 Da in weight, or more typically less than 500 Da.

According to another embodiment the agent is an E2F-1 protein which is arginine-methylation defective. This approach relates directly to the E2F-1 protein whereas the PRMT5 approach has an indirect effect on the E2F-1 protein with equivalent end results.

According to a further embodiment the agent is an antibody or antigen binding fragment thereof, for example an antibody that specifically binds to and blocks the function of PRMT5 or an antibody that specifically binds to arginine-methylated E2F-1 protein, for example an antibody which specifically binds to a peptide comprising a sequence in which one or more of the arginine residues is methylated, for example the sequence RGR(Me)GR(Me) [SEQ ID No.4] or a methylated E2F-1 peptide comprising or consisting of the sequence CESSGPARGR(Me)GR(Me)HPGKG [SEQ ID No.5]. For binding within the cell, the antibody may be an intrabody that is expressed within the target cell. An antibody may also be used to detect E2F-1 methylation and in a method of identifying a proliferative disease which may be susceptible to treatment by the inhibition of PRMT5 and/or stabilisation of intron retention in neoepitopes.

The antibody may be an antibody that specifically binds to arginine-methylated E2F-1 protein. Typically the antibody specifically binds to a methylated E2F-1 peptide comprising or consisting of the sequence RGRGR [SEQ ID No.6] in which one or more of the arginine residues is methylated, for example the sequence RGR(Me)GR(Me) [SEQ ID No.4]. Most typically the antibody specifically binds to a methylated E2F-1 peptide comprising or consisting of the sequence CESSGPARGR(Me)GR(Me)HPGKG [SEQ ID No.5].

The term ‘antibody’ as used herein includes all forms of antibodies such as recombinant antibodies, human or humanized antibodies, chimeric antibodies, single chain antibodies, polyclonal antibodies, monoclonal antibodies etc. Typically the antibody is a monoclonal antibody. The invention is also applicable to antibody fragments and derivatives that are capable of binding to the antigen.

The skilled person could make such antibodies by known methods. Various forms of antibodies can be made using standard recombinant DNA techniques (Winter and Milstein, Nature, 349, pp. 293-99 (1991)). For example, the production of rat proteolytic fragments of IgG antibodies is described by Rousseaux, J (Methods in Enzymology 1986; 121; 663). Antibodies: A Laboratory Manual (Ed Harlow, Edward Harlow, David Lane, CSHL Press, 1988) describes obtaining fragments of human antibodies. Gilliland et al (Tissue Antigens 1996; 47(1):1-20) describes a general method for isolating the variable regions of antibodies and the production of a chimeric antibody. The preparation of monoclonal antibodies is a well-known process (Kohler et al., Nature, 256:495 (1975)).

Chimeric and humanised, e.g. CDR-grafted, antibodies may be used in accordance with the present invention. These antibodies are less immunogenic than the corresponding rodent antibodies. Thus, the antibody may have CDRs which are of different origin to the variable framework region. Similarly, the antibody may have CDRs of different origin to the constant region.

Preferred antibodies according to the present invention are such that the affinity constant for the antigen is 10⁻⁶ mole⁻¹ or more, for example up to 10¹² mole⁻¹. Ligands of different affinities may be suitable for different uses so that, for example, an affinity of 10⁶, 10⁷, 10⁸, 10⁹, 10¹⁰ or 10¹¹ mole⁻¹ or more may be appropriate in some cases. However antibodies with an affinity in the range of 10⁶ to 10⁸ mole⁻¹ will often be suitable.

Antibody affinity is alternatively defined by way of the dissociation constant, Kd. A Kd in at least the micromolar range (10⁻⁶) may be suitable, while a Kd of at least 10⁻⁷, 10⁻⁸ or in the nanomolar (10⁻⁹) range is typical.

Conveniently the antibodies typically also do not exhibit any substantial binding affinity for other antigens, in particular antigens which are not methylated but which otherwise have the same sequence as the desired antigen. Specifically, the antibody typically specifically binds to E2F-1 which is methylated on one or more arginine residues but does not bind to unmethylated E2F-1.

Binding affinities of the antibody and antibody specificity may be tested by assay procedures such as radiolabelled or enzyme labelled binding assays and use of biacore with solid phase ligand. (Bindon, C. I. et al. 1988 Eur. J. Immunol. 18, 1507-1514; Dall'Acqua, W., et al. Biochemistry 35, 9667; Luo et al. J. Immunological Methods 275 (2203) 31-40; Murphy et al. Curr Protoc Protein Sci. 2006 September; Chapter 19: Unit 19.14).

In accordance with the invention, the agent—e.g. the small molecule drug, peptide, protein, antibody or fragment—may be designed to inhibit the activity of PRMT5 or to block the methylation of E2F-1 at one or more of the arginine residues at positions 109, 111 or 113 of the protein. This, in turn, is shown in the Examples to produce a new set of stable retained-intron-containing RNA transcripts. These are expected to lead to RI-containing neoepitopes that can be recognised by the immune system and thus direct the immune system to target the cell expressing them for destruction. Methylated E2F-1 refers to an E2F-1 protein which is methylated on 1 or more of residues R109, R111 and R113. Typically, methylated E2F-1 is di-methylated on two residues of E2F-1 selected from residues R109, R111 and R113. Most typically methylated E2F-1 is di-methylated on residues R111 and R113. Typically the methylation is symmetric.

Alternatively antisense or RNA interference (RNAi) technology may be used to reduce expression of the PRMT5 protein. For example, an antisense molecule, a short hairpin RNA (shRNA) or a small interfering RNA (siRNA) may be administered in accordance with the method of the invention, in order to downregulate expression of PRMT5. Antisense, shRNA and siRNA technologies are known to the skilled person and are described in Goyal 2009, Hajeri 2009, Oh 2009, Singh 2009, Rao 2009, Tilesi 2009, Singer 2008 and Bernards 2006, all of which are incorporated herein by reference.

An antisense molecule typically comprises a single-stranded oligonucleotide of approximately 12 to 20 nucleotides in length, most typically about 16 nucleotides. A small interfering RNA (siRNA) typically comprises a double stranded oligonucleotide of approximately 14 to 22 base pairs in length, most typically 16 to 20 base pairs in length. For example the siRNA may comprise a double stranded olionucleotide of 16, 17, 18, 19 or 20 base pairs in length. The siRNA may have a single or double overhang at one or both ends, i.e. a 3′ or 5′ overhang consisting of one or two bases.

An antisense or siRNA molecule may be designed based on the mRNA sequence that encodes the target protein, namely the PRMT5 enzyme. Typically the antisense or siRNA molecule is complementary to a section of the mRNA sequence that encodes the target protein. The design of siRNA molecules is known in the art (e.g. Tilesi 2009). A series of oligonucleotides may be designed and tested against the target mRNA (e.g. PRMT5 mRNA) and a reduction in the amount of target mRNA looked for. The levels of mRNA can be measured directly or indirectly by monitoring PRMT5 protein levels, by known techniques.

A typical siRNA for use in accordance with the invention comprises the sequence 5′ CCGCUAUUGCACCUUGGAA [SEQ ID No.2] or a sequence with at least 60%, at least 70%, at least 80%, at least 90%, at least 95% or at least 99% identity thereto. Another preferred siRNA for use in accordance with the invention comprises the sequence 5′ CAACAGAGAUCCUAUGAUU [SEQ ID No.3] or a sequence with at least 60%, at least 70%, at least 80%, at least 90%, at least 95% or at least 99% identity thereto. Another preferred siRNA, for inhibiting E2F1 is 5′-CUCCUCGCAGAUCGUCAUCUU-3′ [SEQ ID No.7], or a sequence with at least 60%, at least 70%, at least 80%, at least 90%, at least 95% or at least 99% identity thereto.

An ‘oligonucleotide’ or ‘oligo’ shall mean multiple nucleotides (i.e. molecules comprising a sugar (e.g. ribose or deoxyribose) linked to a phosphate group and to an exchangeable organic base, which is either a substituted pyrimidine (e.g. cytosine (C), thymine (T) or uracil (U)) or a substituted purine (e.g. adenine (A) or guanine (G)). The term ‘oligonucleotide’ as used herein refers to both oligoribonucleotides and oligodeoxyribonucleotides. The term ‘oligonucleotide’ shall also include oligonucleosides (i.e. an oligonucleotide minus the phosphate) and any other organic base containing polymer. Oligonucleotides can be obtained from existing nucleic acid sources (e.g. genomic or cDNA), but are typically synthetic (e.g. produced by oligonucleotide synthesis).

In preferred embodiments the oligonucleotide may be modified to improve its stability and/or potency. A ‘stabilized oligonucleotide’ shall mean an oligonucleotide that is more resistant to in vivo degradation (e.g. via an exo- or endo-nuclease) than the same oligonucleotide which is not stabilized, for example it may be degraded at least 2, 3, 4 or 5 times more slowly than the non-stabilized oligonucleotide. Preferred stabilized oligonucleotides of the invention have a modified phosphate backbone. Especially preferred oligonucleotides have a phosphorothioate modified phosphate backbone (i.e. at least one of the phosphate oxygens is replaced by sulfur). Other stabilized oligonucleotides include: nonionic DNA analogs, such as alkyl- and aryl-phosphonates (in which the charged phosphonate oxygen is replaced by an alkyl or aryl group), phosphodiester and alkylphosphotriesters, in which the charged oxygen moiety is alkylated.

Oligonucleotides which contain a diol, such as tetraethyleneglycol or hexaethyleneglycol, at either or both termini have also been shown to be substantially resistant to nuclease degradation.

Other ways to increase the stability and/or potency of the oligonucleotide include 2′-O modifications and/or LNA modifications.

In other preferred embodiments the oligonucleotide may be modified to promote local, topical or systemic delivery. For example, it may be conjugated to an aptamer, formulated in a lipid nanoparticle such as a liposome, polyconjugated, conjugated to a lipophilic molecule such as cholesterol, contained in a cyclodextrin nanoparticle or complexed with an antibody. To promote systemic delivery the oligonucleotide is typically modified in one of these ways. These and other suitable modifications are described by Toudjarska and de Fougerolles 2009.

In one embodiment, the agent is a PRMT5 inhibitor and is a compound of formula I, or a salt, solvate or hydrate thereof, as provided in claim 1 of WO-A-2018/167276. These compounds have the following general structure:

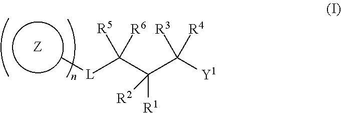

wherein,
Y¹ is a group selected from one of formula A and B,

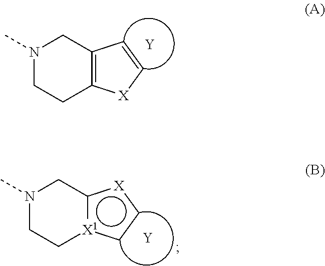

X is selected from O, S, CH and NR⁷;
X1 is selected from C and N;
Y is selected from a fused aryl group and a fused heteroaryl group, where each group is optionally substituted with one or more R11;
n is 1 and L is selected from —(CH2)PN(Ra)C(O)—, —(CH2)PC(O)N(Ra)—, —(CH2)pN(Ra)S(Oq)-, —(CH2)pS(Oq)N(Ra)—, —(CH2)pN(Rb)C(O)N(Rb)—, —(CH2)pN(Rc)C(O)O— and —(CH2)pOC(O)N(Rc)-; or
n is 0 and L is selected from Rd(Re)NC(O)—, —Rd(Re)NC(O)N(Rb)—, Rd(Re)N(Rc)C(O)0-Rd(Re)N(Rc)S(Oq) and Rd(Re)N—;
p is a number selected from 0, 1, 2 and 3;
q is a number selected from 1 and 2;
Z is selected from C6-11 aryl optionally substituted by one or more R10, (C7-16)alkylaryl optionally substituted by one or more R10, C3-11 cycloalkyl optionally substituted by one or more R10, (C4-17)cycloalkylalkyl optionally substituted by one or more R10, 3-15 membered heterocycloalkyl optionally substituted by one or more R10, 4-21 membered alkylheterocycloalkyl optionally substituted by one or more R10, 5-15 membered heteroaryl optionally substituted by one or more R10, and 6-21 membered alkylheteroaryl optionally substituted by one or more R10;
R1 is selected from hydrogen, halogen, —NReRd, ORf, and C1-6 alkyl optionally substituted with one or more R9;
R2 is selected from hydrogen, halogen and C1-6 alkyl optionally substituted with one or more R9;
R3, R4, R5 and R6 are independently selected from hydrogen, halogen and C1-6 alkyl optionally substituted with one or more R9;
R7 is selected from hydrogen, hydroxyl, C1-6 alkyl, C1-6 haloalkyl, phenyl and C3-6 cycloalkyl, wherein said C1-6 alkyl, phenyl and C3-6 cycloalkyl are optionally substituted by one or more substituents selected from hydroxyl, halogen, ═O, CN, CORa, NRaRb, C1-6 haloalkyl, C3-6 cycloalkyl, C6-11 aryl, 3-7 membered heterocycloalkyl, C1-6 alkyl and O—C1-6 alkyl;
each R9 is independently selected from hydrogen, hydroxyl, halogen, CN, C1-6 haloalkyl, 3-7 membered heterocycloalkyl, C3-6 cycloalkyl, C1-6 alkyl, 0-C1-6 alkyl and phenyl, wherein said C1-6 alkyl, phenyl, 3-7 membered heterocycloalkyl and C3-6 cycloalkyl are optionally substituted with one or more groups selected from hydroxyl, ═O, halogen, CN, NRaRb, CORa, C1-6 haloalkyl, C3-6 cycloalkyl, phenyl, 3-7 membered heterocycloalkyl, C1-6 alkyl and O-C1-6 alkyl;
each R10 is independently selected from hydrogen, hydroxyl, ═O, halogen, CN, C1-6 haloalkyl, C1-6 haloalkoxy, C1-6 alkyl, 0-C1-6 alkyl, C3-6 cycloalkyl, phenyl, 5-6 membered heteroaryl, 3-7 membered heterocycloalkyl, —C(═O)Rd, —C(═O)ORd, —C(═O)NReRd, —C(O)C(═O)Rd, —NReRd, —NReC(═O)Rd, —NReC(═O)ORd, —NReC(═O)NReRd, —NReS(═O)2Rd, —NReS(═O)2NReRd, —ORd, —SRd, —OC(═O)Rd, —OC(═O)NReRd, —OC(═O)ORd, —S(═O)2Rd, —S(═O)Rd, —OS(═O)Rd, —OS(═O)2Rd, —OS(═O)2ORd, —S(═O)NReRd, —OS(═O)2NReRd, and —S(═O)2NReRd, where said C3-6 cycloalkyl, C1-6 alkyl, phenyl, 5-6 membered heteroaryl and 3-7 membered heterocycloalkyl are optionally substituted with one or more groups selected from hydroxyl, halogen, ═O, CN, C1-6 haloalkyl, C1-6 haloalkoxy, C3-6 cycloalkyl, C1-6 alkyl and 0-C1-6 alkyl;
R11 is selected from hydrogen, hydroxyl, halogen, CN, NRaRb, C1-6 haloalkyl, 3-7 membered heterocycloalkyl, C3-6 cycloalkyl, C1-6 alkyl, 0-C1-6 alkyl and phenyl, wherein said C1-6 alkyl, phenyl, 3-7 membered heterocycloalkyl and C3-6 cycloalkyl are optionally substituted with one or more groups selected from hydroxyl, ═O, halogen, CN, CORa, NRaRb, C1-6 haloalkyl, C3-6 cycloalkyl, C6-11 aryl, 3-7 membered heterocycloalkyl, C1-6 alkyl and 0-C1-6 alkyl;
each Ra, Rb and Rc is independently selected from hydrogen and C1-6alkyl;
each Rd is independently selected from hydrogen, hydroxyl, halogen, CN, C1-6 haloalkyl, 3-7 membered heterocycloalkyl, C3-6 cycloalkyl, C1-6 alkyl, 0-C1-6 alkyl and C6-n aryl, wherein said C1-6 alkyl, C6-11 aryl, 3-7 membered heterocycloalkyl and C3-6 cycloalkyl are optionally substituted with one or more groups selected from hydroxyl, ═O, halogen, CN, CORa, NRaRb, C1-6 haloalkyl, C3-6 cycloalkyl, C6-11 aryl, 3-7 membered heterocycloalkyl, C1-6 alkyl and O—C1-6 alkyl;
each Re is independently selected from hydrogen, hydroxyl, halogen, CN, C1-6 haloalkyl, C3-6 cycloalkyl, C1-6 alkyl and O-C1-6 alkyl; or
Re and Rd, when attached to the same atom, together with the atom to which they are attached form a 3-7 membered heterocycloalkyl ring, optionally substituted with one or more substituent selected from hydroxyl, ═O, halogen, CN, CORa, NRaRb, C1-6 haloalkyl, C3-6 cycloalkyl, C6-11 aryl, 3-7 membered heterocycloalkyl, C1-6 alkyl and O-C1-6 alkyl; and
Rf is independently selected from hydrogen and C1-6 alkyl optionally substituted with one or more substituents selected from hydroxyl, halogen, CN, CORa, NRaRb, C1-6 haloalkyl, C3-6 cycloalkyl, phenyl, 3-7 membered heterocycloalkyl and O-C1-6 alkyl;
with the proviso that the compound of formula I is not:
2-[3-(N-benzyl-N-methylamino)propyl]-2,3,4,9-tetrahydro-1 H-pyrido[3,4-b] indole;
2-[3-(N-benzyl-N-methylamino)propyl]-1-phenyl-2,3,4,9-tetrahydro-1 H— pyrido[3,4-b]indole;
2-[3-(N-benzyl-N-methylamino)propyl]-1,2,3,4-tetrahydrobenzofuro[3,2-c] pyridine;
2-[3-(N-methyl-N-phenylethylamino)propyl]-2,3,4,9-tetrahydro-1 H-pyrido[3,4-b]indole;
2-[3-(N-methyl-N-phenylethylamino)propyl]-1-phenyl-2,3,4,9-tetrahydro-1 H-pyrido[3,4-b]indole;
2-[3-(N-methyl-N-phenylethylamino)propyl]-1,2,3,4-tetrahydrobenzofuro[3,2-c]pyridine;
2-(3-(pyrrolidin-1 yl] propyl]-2,3,4,9-tetrahydro-1 H-pyrido[3,4-b]indole;
2-(3-(pyrrolidin-1 yl] propyl]-1-phenyl-2,3,4,9-tetrahydro-1 H-pyrido[3,4-b]indole;
2-(3-(pyrrolidin-1 yl] propyl]-1,2,3,4-tetrahydrobenzofuro[3,2-c]pyridine;
2-[3-(isoindolin-2-yl)propyl]-2,3,4,9-tetrahydro-1 H-pyrido[3,4-b]indole;
2-[3-(isoindolin-2-yl)propyl]-1-phenyl-2,3,4,9-tetrahydro-1 H-pyrido[3,4-b]indole;
2-[3-(isoindolin-2-yl)propyl]-1,2,3,4-tetrahydrobenzofuro[3,2-c]pyridine;
1-(3-(8-methoxy-1,3,4,5-tetrahydro-1 H-pyrido[4,3-b]indol-2-yl)propyl)piperazine; or
1-(3\hat{ }1 H,2H,3H,4H,9H-pyrido[3,4-b]indol-2-yl}propyl)piperazine trihydrochloride.

Exemplary agents of this type include the compounds set out in claim 27 of WO-A-2018/167276:
6-(cyclobutylamino)-N-(2-hydroxy-3-{1 H,2H,3H,4H,5H-pyrido[4,3-b]indol-2-yl}propyl)-2-(4-methylpiperazin-1-yl)pyrimidine-4-carboxamide;
2-(cyclobutylamino)-N-(2-hydroxy-3-{1H,2H,3H,4H,5H-pyrido[4,3-b]indol-2-yl}propyl)-6-(4-methylpiperazin-1-yl)pyridine-4-carboxamide;
6-(cyclobutylamino)-N-(2-hydroxy-3-{1H,2H,3H,4H,5H-pyrido[4,3-b]indol-2-yl}propyl)pyrimidine-4-carboxamide;
6-[(1-acetylpiperidin-4-yl)amino]-N-(2-hydroxy-3-{1 H,2H,3H,4H,5H-pyrido[4,3-b]indol-2-yl}propyl)pyrimidine-4-carboxamide;
N-(2-hydroxy-3-{1 H,2H,3H,4H,5H-pyrido[4,3-b]indol-2-yl}propyl)-4-({3-oxa-8-azabicyclo[3.2.1]octan-8-yl}carbonyl)benzamide;
6-(cyclobutylamino)-N-(2-hydroxy-3-{5-methyl-1 H,2H,3H,4H,5H-pyrido[4,3-b]indol-2-yl}propyl)-2-(4-methylpiperazin-1-yl)pyrimidine-4-carboxamide;
2-(cyclobutylamino)-N-(2-hydroxy-3-{5-methyl-1 H,2H,3H,4H,5H-pyrido[4,3-b]indol-2-yl}propyl)-6-(4-methylpiperazin-1-yl)pyridine-4-carboxamide;
N-(2-hydroxy-3-{5-methyl-1 H,2H,3H,4H,5H-pyrido[4,3-b]indol-2-yl}propyl)-4-({3-oxa-8-azabicyclo[3.2.1]octan-8-yl}carbonyl)benzamide;
6-(cyclobutylamino)-N-(2-hydroxy-3-{1 H,2H,3H,4H,9H-pyrido[3,4-b]indol-2-yl}propyl)-2-(4-methylpiperazin-1-yl)pyrimidine-4-carboxamide;
2-(cyclobutylamino)-N-(2-hydroxy-3-{1 H,2H,3H,4H,9H-pyrido[3,4-b]indol-2-yl}propyl)-6-(4-methylpiperazin-1-yl)pyridine-4-carboxamide;
6-(Cyclobutylamino)-N-(2-hydroxy-3-{1 H,2H,3H,4H,9H-pyrido[3,4-b]indol-2-yl}propyl)pyrimidine-4-carboxamide;
6-[(1-acetylpiperidin-4-yl)amino]-N-(2-hydroxy-3-{1 H,2H,3H,4H,9H-pyrido[3,4-b]indol-2-yl}propyl)pyrimidine-4-carboxamide;
N-(2-hydroxy-3-{1 H,2H,3H,4H,9H-pyrido[3,4-b]indol-2-yl}propyl)-4-[(morpholin-4-yl)carbonyl]benzamide;
N-(2-hydroxy-3-{1 H,2H,3H,4H,9H-pyrido[3,4-b]indol-2-yl}propyl)-4-({3-oxa-8-azabicyclo[3.2.1]octan-8-yl}carbonyl)benzamide;
6-(cyclobutylamino)-N-(2-hydroxy-3-{9-methyl-1 H,2H,3H,4H,9H-pyrido[3,4-b]indol-2-yl}propyl)-2-(4-methylpiperazin-1-yl)pyrimidine-4-carboxamide;
2-(cyclobutylamino)-N-(2-hydroxy-3-{9-methyl-1 H,2H,3H,4H,9H-pyrido[3,4-b]indol-2-yl}propyl)-6-(4-methylpiperazin-1-yl)pyridine-4-carboxamide;
6-(cyclobutylamino)-N-(2-hydroxy-3-{9-methyl-1 H,2H,3H,4H,9H-pyrido[3,4-b]indol-2-yl}propyl)pyrimidine-4-carboxamide;
6-[(1-acetylpiperidin-4-yl)amino]-N-(2-hydroxy-3\hat{ }9-methyl-1 H,2H,3H,4H,9H-pyrido[3,4-b]indol-2-yl}propyl)pyrimidine-4-carboxamide;
N-(2-hydroxy-3-{9-methyl-1 H,2H,3H,4H,9H-pyrido[3,4-b]indol-2-yl}propyl)-4-({3-oxa-8-azabicyclo[3.2.1]octan-8-yl}carbonyl)benzamide;
2-hydroxy-3-(1,3,4,9-tetrahydro-2H-pyrido[3,4-b]indol-2-yl)propyl 3-phenylpyrrolidine-1-carboxylate;
2-Hydroxy-3-(1,3,4,9-tetrahydro-2H-pyrido[3,4-b]indol-2-yl)propyl 3,4-dihydroisoquinoline-2(1 H)-carboxylate;
N-(2-hydroxy-3-(1,3,4,9-tetrahydro-2H-pyrido[3,4-b]indol-2-yl)propyl)-3,4-dihydroisoquinoline-2(1 H)-carboxamide;
N-(2-hydroxy-3-(1,3,4,9-tetrahydro-2H-pyrido[3,4-b]indol-2-yl)propyl)-3-phenyl pyrrolidine-1-carboxamide;
N-(2-hydroxy-3-(1,3,4,9-tetrahydro-2H-pyrido[3,4-b]indol-2-yl)propyl)-3-phenylpiperidine-1-carboxamide;
6-((1-acetylpiperidin\hat{ }-yl)amino)-N-(3-(1,3,4,9-tetrahydro-2H-pyrido[3,4-b]indol-2-yl)propyl)pyrimidine-4-carboxamide;
(S)-6-((1-acetylpiperidin-4-yl)amino)-N-(2-hydroxy-3-(1,3,4,9-tetrahydro-2H-pyrido [3,4-b]indol-2-yl)propyl)pyrimidine-4-carboxamide;
(R)-6-((1-acetylpiperidin-4-yl)amino)-N-(2-hydroxy-3-(1,3,4,9-tetrahydro-2H-pyrido [3,4-b]indol-2-yl)propyl)pyrimidine-4-carboxamide; and
6-((1-acetylpiperidin-4-yl)amino)-N-(3-(3,4-dihydropyrazino[1,2-a]indol-2(1H)-yl)-2-hydroxypropyl)pyrimidine-4-carboxamide.

In another embodiment, the agent is a PRMT5 inhibitor that is a compound of formula I, or a salt, solvate or hydrate thereof, as provided in claim 1 of WO-A-2018/167269. These compounds have the following general structure:

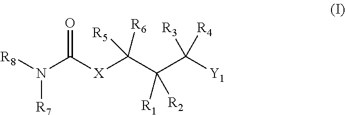

wherein,

R₁, R₃, R₄, R₅ and R₆ are each independently selected from hydrogen and C₁₋₃ alkyl;

R₂ is selected from hydrogen and R₁₄;

X is O or NR₉, where R₉ is hydrogen or a C₁₋₃ alkyl;

Y₁ is a group selected from one of formula A and B,

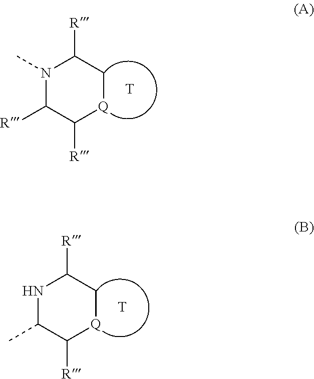

where each R′″ is independently selected from H or C1-3 alkyl;
Q is C or N;
T is selected from a fused phenyl group and a fused 5- or 6-membered heteroaryl group, wherein each group is optionally substituted with one or more substituents selected from halo and C1-3 alkyl; and
R7 and R8 are taken together with the intervening nitrogen atom to form a 3-12 membered heterocycloalkyl ring, wherein the 3-12 membered heterocycloalkyl ring is optionally substituted with one or more R10; and/or optionally fused to one or more C6-12 aryl, C5-12 heteroaryl, C3-8 cycloalkyl and 3-12 membered heterocycloalkyl rings, wherein each fused C6-i2 aryl, C5-12 heteroaryl, C3-8 cycloalkyl and 3-12 membered heterocycloalkyl ring is optionally substituted with one or more R14;
R10 is selected from a group of the formula L1-L2-R11 or L2-L1-R11, where is a linker of the formula —[CR12R13]n-, where n is an integer of from 0 to 3 and R12 and R13 are in each instance each independently selected from H or C1 to C2 alkyl,
where L2 is absent or a linker that is selected from O, S, SO, SO2, N(R′), C(O), C(O)O, [O(CH2)r]s, [(CH2)rO]s, OC(O), CH(OR′), C(O)N(R′), N(R′)C(0), N(R′)C(0)N(R′), S02N(R′) or N(R′)S02, where R and R″ are each independently selected from hydrogen and a Ci to C2 alkyl, and where r is 1 or 2 and s is 1 to 4,
R11 is independently selected from hydrogen, CN, NO2, hydroxyl, =0, halogen, C1-6 haloalkyl, C1-6 haloalkoxy, C1-6 alkyl, 0-C1-6 alkyl, C3-6 cycloalkyl, C6-12 aryl, C5-12 heteroaryl, 3-10 membered heterocycloalkyl, —C(=0)Rd, —C(=0)ORd, —C(=0)NReRd, —C(O)C(=0)Rd, —NReRd, —NReC(=0)Rd, —NReC(=0)ORd, —NReC(=0)NReRd, —NReS(=0)2Rd, NReS(=0)2NReRd, —ORd, —SRd, —OC(=0)Rd, —OC(=0)NReRd, —OC(=0)ORd, —S(=0)2Rd, —S(=0)Rd, —OS(=0)Rd, —OS(=0)2Rd, —OS(=0)2ORd, —S(=0)NReRd, —OS(=0)2NReRd, and —S(═O)2NReRd, wherein, where R11 is independently selected from C3-6 cycloalkyl, C6-12 aryl, C5-12 heteroaryl and 3-10 membered heterocycloalkyl, each C3-6 cycloalkyl, C6-12 aryl, C5-12 heteroaryl and 3-10 membered heterocycloalkyl is optionally substituted with one or more R14;
each Ra and Rb is independently selected from hydrogen and C1-6 alkyl;
each Rd is independently selected from hydrogen, hydroxyl, halogen, CN, C1-6 haloalkyl, 3-7 membered heterocycloalkyl, C3-6 cycloalkyl, C1-6 alkyl, 0-Ci-6 alkyl and C6-n aryl, wherein said C1-6 alkyl, C6-11 aryl, 3-7 membered heterocycloalkyl and C3-6 cycloalkyl are optionally substituted with one or more groups selected from hydroxyl, =0, halogen, CN, CORa, NRaRb, C1-6 haloalkyl, C3-6 cycloalkyl, C6-11 aryl, 3-7 membered heterocycloalkyl, C1-6 alkyl and O—C1-6 alkyl;
each Re is independently selected from hydrogen, hydroxyl, halogen, CN, C1-6 haloalkyl, C3-6 cycloalkyl, C1-6 alkyl and O-C1-6 alkyl; or
Re and Rd, when attached to the same atom, together with the atom to which they are attached form a 3-7 membered heterocycloalkyl ring, optionally substituted with one or more substituent selected from hydroxyl, =0, halogen, CN, CORa, NRaRb, C1-6 haloalkyl, C3-6 cycloalkyl, C6-11 aryl, 3-7 membered heterocycloalkyl, C1-6 alkyl and O-C1-6 alkyl;
and
R14 is independently selected from halo, CN, NO2, hydroxyl, =0, halogen, C1-6 haloalkyl, C1-6 haloalkoxy, C1-6 alkyl, 0-C1-6 alkyl, C3-6 cycloalkyl, C6-12 aryl, 5-6 membered heteroaryl, 3-7 membered heterocycloalkyl, C1-6alkylC6-12aryl, —C(=0)Rd, —C(=0)ORd, —C(=0)NReRd, —C(O)C(=0)Rd, —NReRd, —NReC(=0)Rd, —NReC(=0)ORd, —NReC(=0)NReRd, —NReS(=0)2Rd, —NReS(=0)2NReRd, —ORd, —SRd, —OC(=0)Rd, —OC(=0)NReRd, —OC(=0)ORd, —S(=0)2Rd, —S(=0)Rd, —OS(=0)Rd, —OS(=0)2Rd, —OS(=0)2ORd, —S(=0)NReRd, —OS(=0)2NReRd, and —S(=0)2NReRd.

Exemplary agents of this type include the compounds set out in claim 29 of WO-A-2018/167276 or a salt, solvate or hydrate thereof, which include the following:

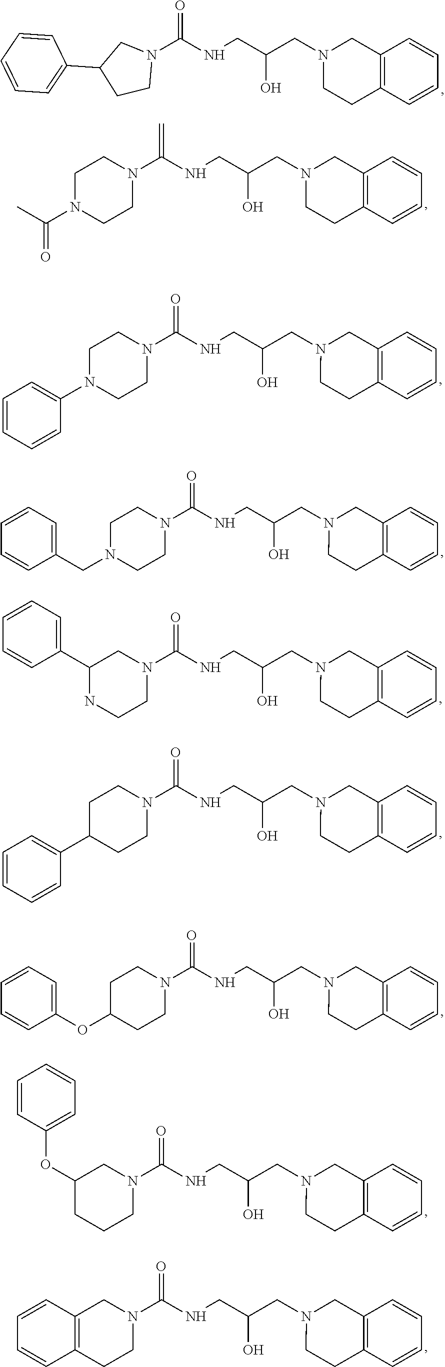

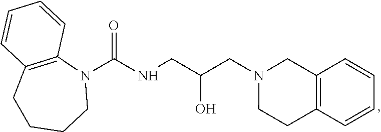 [SMILES: O=C(NCC(O)CN1CCc2ccccc2C1)N1CCCCc2ccccc21]

In an alternative embodiment of the invention, a gene therapy approach may be taken, in which an E2F-1 protein which is arginine-methylation defective is provided. The sequence information for E2F-1 may be found at the National Center for Biotechnology Information (http://www.ncbi.nlm.nih.gov/pubmed/) under accession numbers NM_005225 (nucleotide) and NP_005216 (protein). The human protein sequence is reproduced here as SEQ ID No.8:

| | |
|---|---|
| 1 | MALAGAPAGG PCAPALEALL GAGALRLLDS SQIVIISAAQ |
| | DASAPPAPTG PAAPAAGPCD |
| 61 | PDLLLFATPQ APRPTPSAPR PALGRPPVKR RLDLETDHQY |
| | LAESSGPARG RGRHPGKGVK |
| 121 | SPGEKSRYET SLNLTTKRFL ELLSHSADGV VDLNWAAEVL |
| | KVQKRRIYDI TNVLEGIQLI |
| 181 | AKKSKNHIQW LGSHTTVGVG GRLEGLTQDL RQLQESEQQL |
| | DHLMNICTTQ LRLLSEDTDS |
| 241 | QRLAYVTCQD LRSIADPAEQ MVMVIKAPPE TQLQAVDSSE |
| | NFQISLKSKQ GPIDVFLCPE |
| 301 | ETVGGISPGK TPSQEVTSEE ENRATDSATI VSPPPSSPPS |
| | SLTTDPSQSL LSLEQEPLLS |
| 361 | RMGSLRAPVD EDRLSPLVAA DSLLEHVRED FSGLLPEEFI |
| | SLSPPHEALD YHFGLEEGEG |
| 421 | IRDLFDCDFG DLTPLDF |

An E2F-1 protein which is arginine-methylation defective is mutated so that one or more of residues R109, R111 and R113 (underlined above) are substituted with residues that are resistant to methylation. Typically the arginine (R) residue(s) are substituted with lysine (K) residue(s). Typically the methylation-defective E2F-1 protein contains the mutations R111K and R113K (the ‘KK mutant’) or R109K, R111K and R113K (the ‘KKK mutant’). Accordingly the invention also provides a methylation-defective E2F-1 protein for the treatment of a proliferative disease and the use of a methylation-defective E2F-1 protein for the manufacture of a medicament for the treatment of a proliferative disease. Further the invention provides an oligonucleotide which encodes a methylation-defective E2F-1 protein for the treatment of a proliferative disease and the use of an oligonucleotide which encodes a methylation-defective E2F-1 protein for the manufacture of a medicament for the treatment of a proliferative disease.

### Treatment of Proliferative Diseases Such as Cancer

The methods and agents of the invention may be used in the treatment of a proliferative disease. As such, the methods may ultimately result in the killing of cells which proliferate abnormally, such as cancerous cells, including tumour cells, and other (non-malignant) tumour cells.

The therapeutic agent may be the agent that regulates the neoantigen production, for example a PRMT5 inhibitor. The therapeutic agent may alternatively be one or more epitopes that have been identified in or on a cancer cell following stabilisation of the retained introns using such an agent. The neoepitopes have particular utility as a cancer vaccine, which in some embodiments may be autologous to the patient from whom the neoepitopes were identified.

In some embodiments, the therapy may be effected by a method that comprises the steps of:
(a) administering an agent that regulates neoantigen production to the cancer patient;
(b) identifying retained introns in RNA transcripts from the cancer patient to whom the agent has been administered, for example from a tumour biopsy obtained from the patient;
(c) identifying one more neo-antigens that result from the retained introns.

This method can optionally contain one or more steps, such as:
(d) producing proteins or peptides comprising the identified neoantigens,
(e) formulating the identified neoantigen(s) into a pharmaceutical composition and/or
(f) administering to a patient one or more of the identified neoantigens.

Accordingly, the invention includes a method of treating or preventing cancer in a patient using a composition of the invention. The term “patient” includes human and other mammalian subjects that receive either prophylactic or therapeutic treatment.

The methods may be particularly effective in targeting cancer cells that contain methylated E2F-1, in particular high levels of E2F-1 methylation. In such cells, reduction of that methylation has been shown to increase intron retention and neoepitope expression. Thus, the invention provides methods for increasing intron-retained neoepitope expression.

High E2F-1 methylation may be defined by pathologists upon examining a biological sample (e.g. a tumour biopsy) from a patient. For example, a biological sample may be examined and given a total staining score (TSS). Total staining score is a parameter which is well-known to the skilled person. It is based upon the number of cells stained by an antibody specific for the antigen (in this case, methylated E2F-1 protein), the intensity of the staining in the cells and the overall pathology of the tumour (e.g. for bowel cancer the overall pathology may be defined as Dukes' stage A, B, C or D). A high level of E2F-1 methylation may then be defined as a TSS of at least 50%, typically at least 60% or at least 70% or at least 80%.

The term ‘proliferative disease’ as used herein refers to both cancer and non-cancer disease. Typically the proliferative disease is one characterized by neoepitope expression in afflicted patients. The invention can be used to screen for risk of and/or treat a variety of different types of cancer, particularly malignant (and typically solid) tumours of epithelial or mesenchymal cells, e.g. an advanced solid tumour as disclosed in WO-A-02/66019. Examples of cancers that can be screened for risk of and/or treated by the present invention include brain and other central nervous system tumours (e.g. tumours of the meninges, brain, spinal cord, cranial nerves and other parts of central nervous system, e.g. glioblastomas or medulla blastomas); head and/or neck cancer; breast tumours; circulatory system tumours (e.g. heart, mediastinum and pleura, and other intrathoracic organs, vascular tumours and tumour-associated vascular tissue); excretory system tumours (e.g. kidney, renal pelvis, ureter, bladder, other and unspecified urinary organs); gastrointestinal tract tumours (e.g. oesophagus, stomach, small intestine, colon, colorectal, rectosigmoid junction, rectum, anus and anal canal), tumours involving the liver and intrahepatic bile ducts, gall bladder, other and unspecified parts of biliary tract, pancreas, other and digestive organs); head and neck; oral cavity (lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx); reproductive system tumours (e.g. vulva, vagina, Cervix uteri, Corpus uteri, uterus, ovary, and other sites associated with female genital organs, placenta, penis, prostate, tests, and other sites associated with male genital organs); respiratory tract tumours (e.g. nasal cavity and middle ear, accessory sinuses, larynx, trachea, bronchus and lung, e.g. small cell lung cancer or non-small cell lung cancer); skeletal system tumours (e.g. bone and articular cartilage of limbs, bone articular cartilage and other sites); skin tumours (e.g. malignant melanoma of the skin, non-melanoma skin cancer, basal cell carcinoma of skin, squamous cell carcinoma of skin, mesothelioma, Kaposi's sarcoma); and tumours involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites, leukemias, including hairy cell leukemia, multiple myeloma, chronic lymphocytic leukemia, chronic myeloid leukemia, acute myeloid leukemia and acute lymphocytic leukemia, lymphomas, including non-Hodgkin's lymphoma and Hodgkin's lymphoma. Where hereinbefore and subsequently a tumour, a tumour disease, a carcinoma or a cancer is mentioned, also metastasis in the original organ or tissue and/or in any other location are implied alternatively or in addition, whatever is the location of the tumour and/or metastasis.

Cancers of interest include: Bladder cancer; Breast cancer; CNS cancer, optionally glioma; Liver cancer; Melanoma; Non-small-cell lung cancer; Ovarian cancer; Prostate cancer; and Renal cancer.

In particular embodiments, the cancer may be an oesophageal, pancreatic, gastric or hepatic cancer. More particularly, the cancer may be a carcinoma, optionally a colon carcinoma, an oesophageal carcinoma or a hepatocellular carcinoma. The cancer may alternatively be an adenocarcinoma optionally of the colon, pancreas or stomach. A number of these cancers are exemplified in the Examples and Figures.

Compositions of the invention are therefore useful in treating or preventing cancer. The cancer may, in one embodiment, comprise a liquid tumour. In another embodiment, the cancer may comprise a solid tumour.

Compositions of the invention may also be used to treat or prevent metastatic cancers, for example metastasis of each of the cancers described herein.

The compositions of the invention may also be used to treat a benign (non-cancerous, non-malignant) solid tumour, or a premalignant solid tumour.

In one embodiment, the compositions are used for immune modulation. In one embodiment, the therapy is related to immunomodulation.

The invention also provides a method for treating or preventing cancer comprising administering an effective amount of a composition of the invention, thereby treating or preventing the cancer. The composition may be an agent that alters the neoepitope profile of the cancer cell, such as a PRMT5 inhibitor, or a neoepitope or collection of neoepitopes containing retained introns and optionally identified as described elsewhere herein.

In prophylactic applications, pharmaceutical compositions or medicaments are administered to a patient susceptible to, or otherwise at risk of, a particular disease in an amount sufficient to eliminate or reduce the risk or delay the outset of the disease. In therapeutic applications, compositions or medicaments are administered to a patient suspected of, or already suffering from such a disease in an amount sufficient to cure, or at least partially arrest, the symptoms of the disease and its complications. An amount adequate to accomplish this is defined as a therapeutically- or pharmaceutically-effective dose. In both prophylactic and therapeutic regimes, agents are typically administered in several dosages until a sufficient response has been achieved. Typically, the response is monitored and repeated dosages are given if the response starts to fade.

The agents of the invention may optionally be combined with another therapeutic agent to provide a combination therapy. Two therapeutic agents can optionally be (i) administered together in a single pharmaceutical composition, (ii) administered contemporaneously or simultaneously but separately, or (iii) administered separately and sequentially, e.g. [a] then [b], or [b] then [a]. When the agents are administered separately and sequentially, the duration between the administration of the agents may be one hour, one day, one week, two weeks or more.

Effective doses of the compositions of the present invention, for the treatment of the above described conditions vary depending upon many different factors, including means of administration, target site, physiological state of the patient, whether the patient is human or an animal, other medications administered, and whether treatment is prophylactic or therapeutic. Usually, the patient is a human.

As used herein, the terms “treat”, “treatment”, “treating” and “therapy” when used directly in reference to a patient or subject shall be taken to mean the amelioration of one or more symptoms associated with a disorder, or the prevention or prophylaxis of a disorder or one or more symptoms associated with a disorder. The disorders to be treated include, but are not limited to, cancer. Amelioration or prevention of symptoms results from the administration of the agents of the invention, or of a pharmaceutical composition comprising these agents, to a subject in need of said treatment.

By ‘treating a proliferative disease’ it is intended to include the inhibition of the symptoms of a disease, namely, inhibition or retardation of the progression of the disease; and the alleviation of the symptoms of a disease, namely, regression of the disease or the symptoms, or inversion of the progression of the symptoms. It may also include the prevention of the development of a disease or a symptom from a patient who may have a predisposition of the disease or the symptom but has yet been diagnosed to have the disease or the symptom. By ‘treating cancer’ it is intended to include the inhibition of tumour growth, including the prevention of the growth of a tumour in a subject or a reduction in the growth of a pre-existing tumour in a subject. The inhibition can also be the inhibition of the metastasis of a tumour from one site to another. It is disclosed to treat a tumour exhibiting high levels of PRMT5 protein and low levels of E2F-1 protein with a PRMT5 inhibitor in accordance with the invention, in order to inactivate PRMT5 and thereby activate E2F-1.

In other aspects the invention provides a substance which reduces the expression or activity of the enzyme PRMT5 for the treatment of a proliferative disease, for example cancer, and the use of a substance which reduces the expression or activity of the enzyme PRMT5 for the manufacture of a medicament for the treatment of a proliferative disease, for example cancer. Such substances are described above and may be formulated and administered as described herein.

### Pharmaceutical Compositions

The agent of the invention, and the neoantigen or population of neoantigens that can be identified according to the invention, is useful in therapy and can therefore be formulated as a pharmaceutical composition. A pharmaceutically acceptable composition typically includes at least one pharmaceutically acceptable carrier, diluent, vehicle and/or excipient in addition to the agents of the invention. An example of a suitable carrier is Ringer's Lactate solution. A thorough discussion of such components is provided in Gennaro (2000) Remington: The Science and Practice of Pharmacy. 20th edition, ISBN: 0683306472.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The composition, if desired, can also contain one or more pH buffering agents. The carrier may comprise storage media such as Hypothermosol®, commercially available from BioLife Solutions Inc., USA. Examples of suitable pharmaceutical carriers are described in “Remington's Pharmaceutical Sciences” by E W Martin. Such compositions will contain a prophylactically or therapeutically effective amount of a prophylactic or therapeutic agent typically in purified form, together with a suitable amount of carrier so as to provide the form for proper administration to the subject. The formulation should suit the mode of administration. In a preferred embodiment, the pharmaceutical compositions are sterile and in suitable form for administration to a subject, typically an animal subject, more typically a mammalian subject, and most typically a human subject.

The pharmaceutical composition of the invention may be in a variety of forms. These include, for example, semi-solid, and liquid dosage forms, such as lyophilized preparations, liquid solutions or suspensions, injectable and infusible solutions. The pharmaceutical composition is typically injectable.

The therapeutic agent is administered in vivo in an amount effective to have therapeutic benefit in a patient in need thereof. The term ‘an effective amount’ for purposes of this application shall mean that amount of substance capable of producing the desired effect. In this case, the desired effect may be the slowing of tumour growth, the death of tumour cells, reduction in the size of the tumour, regression of the condition, for example. This is typically achieved by harnessing the immune system to recognise neoepitopes present on cancer cells, which will then be flagged for destruction by the adaptive immune system, for example by or in an immune response comprising CD8+ cytotoxic T lymphocytes. The amount of substance which is given depends upon a variety of factors including the age, weight and condition of the patient, the administration route, the properties of the pharmaceutical composition, the condition of the patient, the judgment of a doctor, the condition and the extent of treatment or prevention desired. The substance may be administered to the individual as a short-term therapy or long-term therapy depending on the condition and the extent of treatment or prevention desired.

It is preferred that the methods, medicaments and compositions of the invention are used for treating cancer, and/or for the treatment, modulation, prophylaxis, and/or amelioration of one or more symptoms associated with cancer.

Pharmaceutical compositions will generally be in aqueous form. Compositions may include a preservative and/or an antioxidant.

To control tonicity, the pharmaceutical composition can comprise a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8 e.g. between 6.5 and 7.5, or between 7.0 and 7.8.

The composition is typically sterile. The composition is typically gluten free. The composition is typically non-pyrogenic.

The pharmaceutical composition can be administered by any appropriate route, which will be apparent to the skilled person depending on the disease or condition to be treated. Typical routes of administration include intravenous, intra-arterial, intramuscular, subcutaneous, intracranial, intranasal or intraperitoneal.

In some embodiments, intratumoral delivery of the therapy may be appropriate. For example, when an oncolytic virus is part of a combination therapy, intratumoral delivery may be suitable to reduce virus dilution and neutralisation. In other embodiments, systemic delivery may be appropriate.

### Selection of T Cells

The invention further provides for the identification of T cells that bind to neoantigen expressed by the cancer, e.g. RI-containing neoantigens. These T cells will typically be identified in the tumour micro-environment. The presence of neo-antigens in a cancer patient, for example following administration of a neoantigen-based vaccine of the invention, should cause an increase in cytotoxic T cells in the tumour microenvironment. This provides an opportunity to harvest these cells, clone and grow them and transfer them back into the patient. This would be similar to CAR-T therapy, but without necessarily having to engineer the cells because they will already be specific for the new neo-antigen generated by the agent-induced RI.

In one embodiment, a patient is administered an agent according to the invention and a personalised neoantigen vaccine prepared and administered. Following administration, the tumour microenvironment is sampled and T cells isolated. These T cells can be screened for affinity to the neoepitopes, and those cells with favourable affinity cultured. The cultured cells can then be administered back to the patient as an autologous T cell therapy. These cells do not require engineering, but may optionally be engineered for example to express a chimeric antigen receptor.

In another embodiment, the tumour microenvironment is sampled after the agent that modifies neoantigens is administered to the patient. In this embodiment, the step of administering a neoepitope vaccine is not required, because the patient may already mount a nascent immune (T cell) response to the neoantigen that is produced or enhanced following administration of the agent. Accordingly, a patient can be administered an agent according to the invention.

Following administration of the agent, the tumour microenvironment is sampled and T cells isolated. These T cells can be screened for affinity to the neoepitopes that are present on the cancer cells (for example by testing for affinity to the cancer cells), and those cells with favourable affinity cultured. The cultured cells can then be administered back to the patient as an autologous T cell therapy. These cells do not require engineering, but may optionally be engineered for example to express a chimeric antigen receptor.

The invention is further described with reference to the following non-limiting examples.

### EXAMPLES

### Arginine Methylation Expands the Regulatory Mechanisms and Extends the Genomic Landscape Under E2F Control.

### Summary—E2F1 Arginine Methylation Influences Alternative Splicing

E2F is a family of master transcription regulators involved in mediating diverse cell fates. Here, we show that residue-specific arginine methylation (meR) by PRMT5 enables E2F1 to regulate many genes at the level of alternative RNA splicing, rather than through its classical transcription-based mechanism. The p100/TSN tudor domain protein reads the meR mark on chromatin-bound E2F1, allowing snRNA components of the splicing machinery to assemble with E2F1. A large set of RNAs including spliced variants associate with E2F1 by virtue of the methyl mark. By focusing on the deSUMOylase SENP7 gene, which we identified as an E2F target gene, we establish that alternative splicing is functionally important for E2F1 activity. Thus, meR E2F1 through selective alternative splicing, enables synthesis of the active SENP7 protein isoform, which through a mechanism affecting HP1 binding enhances E2F target gene activity. Our results reveal an unexpected consequence of arginine methylation, where reader-writer interplay widens the mechanism of control by E2F1, from transcription factor to regulator of alternative RNA splicing, thereby extending the genomic landscape under E2F1 control.

### Introduction

E2F is a family of master transcription regulators involved in mediating diverse cell fates which frequently becomes deregulated in cancer. The retinoblastoma protein (pRb)-E2F pathway is a central player in the control of cell cycle progression in diverse cell-types and its deregulation of primary importance in proliferative disease like cancer, where aberrant pRb activity occurs through a variety of oncogenic mechanisms (1). In the classical view, cyclin-dependent kinases (CDKs) which peak during the G1 phase phosphorylate pRb causing the release of E2F from the pRb/E2F complex, enabling E2F to transcriptionally activate target genes required for cell cycle progression (2-5). E2F1 is one of the most important physiological targets for pRb, and the physical interaction between pRb and E2F1 facilitates transcriptional repression and cell cycle arrest (1, 2). However, E2F1 can foster other biological outcomes, such as the induction of apoptosis (6-8). Understanding the molecular mechanisms responsible for regulating the diverse biological outcomes of E2F1 activity remains a central question in E2F biology which, further, has direct relevance to its pathological role in cancer.

Methylation of arginine side chains is becoming increasingly recognised to be an important protein modification involved with diverse pathways of control (9, 10). In previous studies we identified a small R-rich motif in E2F1 as a target for arginine methylation (11, 12) and uncovered a remarkable relationship between methylation by PRMT5 (symR) and PRMT1 (asymR) in channelling E2F1 through its distinct biological pathways (11, 12); thus, PRMT5-dependent methylation prompts cell growth, in contrast to methylation by PRMT1 which facilitates apoptosis (11, 12). The symR E2F1 mark is read by the tudor domain (TD) protein, p100/TSN (12), which exists as a chromatin-bound symR E2F1 complex on E2F target genes (12, 13). Furthermore, PRMT5-dependent methylation is uniquely relevant to E2F1 amongst the E2F family (11, 12), suggesting that the meR mark is fundamental in the control of E2F1 activity.

Here, we show that methylation by PRMT5 enables E2F1 to regulate a diverse group of genes at the level of alternative RNA splicing, rather than through the classical transcription-based mechanism widely ascribed to E2F1. The impact of E2F1 on alternative RNA splicing requires the tudor domain protein p100/TSN to read the meR mark, allowing components of the splicing machinery such as snRNA to associate with the p100/TSN-E2F1 complex. Consistent with its role in RNA splicing, a large group of RNAs, including spliced intermediates, bind to the E2F1 complex. The majority of genes subject to alternative splicing are poor transcription targets for E2F1. We identified SENP7 as a novel E2F target gene subjected to alternative RNA splicing control by E2F1. At the functional level SENP7 protein influenced E2F target gene activity through regulating chromatin SUMOylation and HP1 binding. Our results reveal an unexpected role for E2F1 in regulating the alternative RNA splicing machinery which occurs through a meR mark-dependent reader-writer interplay, enabling E2F1 to broaden its influence to genes which otherwise are poor transcription targets. The methyl mark therefore confers a new mechanism of control and extends the genomic landscape under E2F1 control.

The inventor has observed experimentally that the retained intron effect is also provided by HDAC inhibitors (data not shown).

### Results

### meR Marks on E2F1 Confer Genome-Wide Effects

To clarify the role of the meR mark in regulating E2F1 activity, we developed a panel of Tet-On inducible cell lines (FIG. 1C Panel A). Each cell line expressed wild-type (WT) E2F1 or its derivative KK (with mutated symR sites at R111 and R113) previously established to be defective in PRMT5 methylation and to exert apoptosis more efficiently than WT E2F1 (12). For comparison, we prepared a cell line expressing R109K (with a mutated asymR site at R109) which cannot be methylated by PRMT1 but retains the PRMT5 symR sites; the R109K derivative is endowed with greater proliferation activity relative to WT E2F1 (12). The induced WT, KK and R109K E2F1 proteins behaved as expected; upon expression each ectopic protein underwent nuclear accumulation, by ChIP localised to the promoter region of E2F target genes (FIGS. 7A and 7B) and exhibited similar binding and cellular activities as described previously (FIGS. 7C and 7D) (12). FIG. 1C Panel A indicates the sequences of residues 107 to 115 of WT E2F1 (SEQ ID No.12), R109K E2F1 (SEQ ID No.13) and R111K/R113K (SEQ ID No.14).

We employed RNA-seq to assess the global transcript profile in each stable cell line. Mining the RNA-seq data set for transcripts regulated 2-fold or more upon E2F1 expression (compared to the empty pTRE vector cell line) identified a large number, the majority (around 50% for each cell line) being derived from E2F target genes (FIG. 7E and Table S1), where an E2F target gene was defined by the presence of one or more E2F binding site consensus motifs in the proximal promoter region [−900 to +100] (14). For the WT E2F1 expression condition, 900 E2F target gene transcripts were identified whose expression was affected by more than 2-fold (FIGS. 1C Panel B and 7E), a figure broadly in line with previous reports on E2F transcription targets (15).

Within the population of 2-fold regulated transcripts, the majority were up-regulated although a significant proportion were also down-regulated (70% compared to 30% respectively; FIG. 7E). Further, although a high proportion of up and down-regulated transcripts were shared between the WT and KK E2F1 cell lines (80.7% and 83.5% respectively) (FIG. 1C Panel B) some of the transcripts were non-over-lapping and therefore independently regulated by either WT or KK E2F1 expression. 17.1% and 14.9% of the transcripts were differentially upregulated by either WT or KK respectively, and conversely 24.2% and 20.4% were differentially down-regulated (FIG. 1C Panel B).

A similar analysis was performed on the R109K expression condition. In contrast to WT or KK E2F1, the R109K derivative was less able to influence transcription (FIG. 1C Panel B and 7E); notably, R109K was about 60% as efficient as WT E2F1 in regulating transcription. Further, 93% of the transcripts up-regulated by R109K were shared with WT or KK E2F1, with only 26 unique transcripts detected in the R109K expression condition (FIG. 1C Panel B). A similar pattern was observed when down-regulated transcripts were analysed; again, 86% of the transcripts down-regulated by R109K were shared with WT and KK E2F1, with 24 unique transcripts apparent (FIG. 1C Panel B and 7E). Thus R109K, which retains intact residues R111/R113 methylated by PRMT5, is less able to regulate transcription than its WT and KK counterparts.

We assessed the gene sets which were present in the RNA-seq by Gene Set Analysis (GSA). There were a number of shared gene sets enriched in each condition, including E2F targets (as expected), whereas gene sets connected with the epithelial-mesenchymal transition and hypoxia were generally down-regulated in each condition (FIG. 8A).

It was important to validate the results from the RNA-seq. We therefore measured the expression of a number of E2F target candidate genes identified in the RNA-seq data set where there was evidence for differential expression patterns. For example, LRRC4, ETV1 and FGF4 transcripts were expressed at high levels in the KK cell line, with reduced expression in the R109K cell line, and a similar pattern of expression was evident when transcription from each gene was individually measured in each cell line (FIG. 7F). Conversely, at the global level, KCNIP showed higher expression in R109K compared to KK, and a similar expression pattern was apparent when gene expression was individually measured (FIG. 7F). Moreover, we confirmed that expression of each candidate gene was dependent on E2F1, as silencing endogenous E2F1 with siRNA caused reduced expression of each gene (FIG. 7G).

### E2F1 Permits Alternative RNA Splicing of E2F Target Genes

It was noteworthy that the R109K derivative exhibits a reduced ability to affect transcription (FIG. 1C Panel B and 7E). Because p100/TSN interacts with the splicing machinery (16), and since R109K binds to p100/TSN through PRMT5-dependent methylation of residues R111 and R113 (12), we reasoned that p100/TSN may confer on E2F1 the ability to control RNA splicing. We therefore mined each RNA-seq data set for evidence of alternative RNA splicing using the rMATS algorithm (17) (FIG. 2A). Of great interest was the fact that a large number of transcripts derived from 1560 genes were present in the data set where 1021 (namely 65%; FIG. 2B), identified as E2F target genes, exhibited alternative splicing effects dependent on E2F1 expression.

We observed alternative splicing events in E2F gene transcripts which included skipped exons (SE), alternative 3′ (A3SS) or 5′ (A5SS) splice sites, mutually exclusive exons (MXE) and retained introns (RI) (FIG. 2C and FIG. 8B). Some transcripts were subject to different alternative splicing events (Table S2). Dramatically, although defined as E2F target genes by the presence of canonical E2F DNA binding motifs and cross-referencing to ChIP-seq data sets in ENCODE (ENCODE project consortium, 2012), we found upon mining the RNA-seq data that the vast majority of alternatively-spliced E2F transcripts were modest transcription targets for E2F1 (transcriptionally regulated less than 2-fold upon the expression of E2F1; FIGS. 2D and 2E). Only 42 genes in the transcriptionally up-regulated E2F target gene group, and 17 genes in the down-regulated group, were shared with the alternatively spliced set (less than 3% overlap between the two sets of genes); the majority of E2F1-dependent alternative RNA splicing thus occurred on genes that are poor transcription targets for E2F1 (FIGS. 2D and 2E). The results highlight two categories of E2F target genes, one made up of genes which are good transcription targets (regulated greater than 2-fold by E2F1) and the other composed of genes principally regulated through alternative splicing which, generally, are poor transcription targets.

When each set of alternatively spliced transcripts derived from E2F target genes was compared across the WT, KK and R109K E2F1 expression conditions, qualitative and quantitative differences in the alternatively spliced RNA were apparent, with events that were both shared and unique (FIG. 2A). WT and KK cell lines shared 41% and 36% of the alternatively spliced genes, whilst WT and R109K shared 30% and 34%, and KK and R109K shared 28% and 34% (FIG. 2B ii), highlighting the fact that each E2F1 derivative affects alternative splicing of an overlapping set of RNAs. Further, R109K caused the strongest splicing effect (by rMATS analysis) contrasting with KK, which was least efficient (FIG. 8B). This situation contrasted with the transcription analysis of the RNA-seq data (FIG. 1C Panel B), where R109K was less effective than the WT and KK derivatives in causing differential gene expression.

We performed gene ontology (GO) analysis on the E2F gene sets from which the alternatively spliced transcripts were derived (FIG. 9). Although there was considerable overlap in the GO terms enriched in each condition, such as cellular processes linked to cell cycle, there were a number of marked differences. For example, DNA damage related terms were prevalent under KK expression conditions, whilst catabolic and biosynthetic terms were enriched upon R109K expression.

We also studied the expression level of a variety of E2F target genes connected with splicing, many encoding components of the splicing machinery (Table S3). From an analysis of the RNA-seq data, none of the genes were expressed at a significantly different level between the WT, KK or R109K expression conditions (Table S3). The increased level of alternative splicing identified by rMATS therefore cannot be easily attributed to coincident changes in the expression of splicing components.

### Chromatin-Associated E2F1 Binds to Components of the Splicing Machinery

We reasoned that the impact of E2F1 on alternative RNA splicing could be mediated by meR E2F1 interacting with components of the splicing machinery, since the meR reader protein p100/TSN functions in spliceosome assembly and enhances splicing activity (16). We therefore addressed whether small nuclear (sn) RNAs, essential components of the spliceosome (18), could associate with E2F1. By RNA immunoprecipitation (RIP), we found that U1, U4, U5 and U6 snRNA associate with E2F1 in a variety of cell types, including U2OS, HCT116 and MCF7 cells (FIG. 3A to F). Significantly, the interaction of snRNA with E2F1 was dependent on p100/TSN and PRMT5 activity, as it was reduced in cells treated with p100/TSN siRNA (FIG. 10A), and absent in cells treated with the PRMT5 inhibitor EPZ015666 (19) (FIGS. 3B, E and F). To assess whether snRNA binding to E2F1 required an intact DNA binding domain and therefore was likely to occur with chromatin-associated E2F1, we prepared E2F1 derivatives with compromised DNA binding activity (FIG. 3G). By chromatin immunoprecipitation (ChIP), neither L132E nor R166H bound to chromatin relative to wild-type E2F1 (FIG. 3G), even though each mutant derivative could undergo nuclear accumulation (FIG. 10B). Significantly, upon RIP analysis with L132E or R166H, the level of U6 snRNA was reduced, in contrast to the wild-type (WT) E2F1 RIP where U6 was clearly detectable (FIG. 3H), arguing that the interaction with snRNA occurs with chromatin-bound E2F1.

E2F1 Interacts with a Diverse Set of Alternatively Spliced Transcripts

Having established that arginine methylation and its reader p100/TSN enables E2F1 to influence alternative splicing (FIG. 2) and further allows binding to snRNA, we went on to explore whether any additional RNA species could associate with p100/TSN-E2F1 using RIP-seq. We performed the E2F1 RIP-seq analysis in the presence and absence of p100/TSN, in order to characterise the population of RNA that bound to E2F1 in a meR-p100/TSN-dependent fashion. We observed a large set of RNAs, 384 in total, in the E2F1 RIP-seq that were dependent on the presence of p100/TSN (Table S4). Some of the p100/TSN-dependent RNAs identified in the E2F1 RIP-seq were highlighted to be alternatively spliced RNAs in the rMATS splicing analysis of the RNA-seq data set (Table S5 and FIG. 2A). For example, the lysine acetyl-transferase KAT2B (ΔΨ=0.171-0.232), the lysine methyl-transferase SETD2 (ΔΨ=−0.888), and max dimer protein MGA (ΔΨ=0.436) were identified as alternatively spliced transcripts by rMATs (Table S2).

We further mined the E2F1 RIP-seq data set to identify peak sequencing reads which span exon junctions across the different RNAs which were then related to genomic organisation of the parent gene, enabling us to identify spliced RNA variants. We identified a sub-group of the 384 RNA species where the sequencing reads spanned 27 exon junctions, which correspond to 26 different transcripts derived from 18 genes (Table S6). For example, multiple alternatively spliced transcripts derived from SENP7, MECOM, P3H2, and SPG21 genes were identified in the E2F1 RIP-seq (FIGS. 4A and 5A, and FIGS. 10C and 10D) with similar alternative splicing events apparent in the RNA-seq data (Sashimi plots shown for SENP7 and MECOM; FIG. 10E).

We chose as representative examples and characterised in greater detail SENP7 and MECOM. SENP7 (SUMO1/sentrin specific peptidase 7) is a de-SUMOylase that is involved with control of protein stability, chromatin and transcription (20-22). The SENP7 alternatively spliced RNA variant identified in the RIP-seq, V5, spanned exon junctions 4 and 7, and thus lacked exons 5 and 6 (FIG. 4A). Consistent with the RIP-seq results, the V5 RNA variant was detected in the E2F1 RIP, contrasting with the other SENP7 RNA spliced variants (FIG. 4B). Additionally, the presence of SENP7 V5 variant in the RIP was dependent on E2F1 and p100/TSN, as it was absent upon silencing either E2F1 or p100/TSN protein (FIG. 4B). Further, inhibiting PRMT5 activity with EPZ015666 reduced the interaction between E2F1 and V5 RNA in cells (FIG. 4C), which also coincided with a lower level of the RNA variant (in contrast to the other SENP7 RNA variants which increased) in cells (U2OS and HCT116) treated with EPZ015666 (FIGS. 4D and 4E).

To confirm that SENP7 is a target gene for E2F1, we inspected the genomic DNA sequence around the promoter region (−2 kb to +1 kb) and identified an intronic E2F DNA binding site motif within 450 base pairs of the transcription start site, after the first exon (FIG. 4F). By ChIP, this region of the SENP7 gene was capable of binding E2F1 (FIG. 4F). Moreover, using CRISPR cell lines which lacked E2F1 or p100/TSN, we confirmed that the SENP7 ChIP activity is dependent on E2F1 and is influenced by the presence of p100/TSN (FIG. 4F). These results highlight a role for PRMT5, E2F1 and p100/TSN in directing alternative splicing of SENP7.

We performed a similar analysis of MECOM, which encodes a zinc finger transcription factor involved with different signalling pathways (23). The major MECOM RNA species identified in the RIP-seq was the V7 spliced variant (FIG. 5A). We subsequently verified binding of the V7 RNA variant to E2F1 in diverse cell types (U2OS, HCT116 and MCF7) and the dependency on PRMT5 activity for the RNA interaction with E2F1 (FIG. 5B). By ChIP, an E2F binding site was identified within the first intron of the V7 transcript variant (FIG. 5C) and alternative splicing of MECOM RNA in cells was altered upon PRMT5 inhibition (FIG. 5D). Most significantly, we examined whether MECOM V7 was present in human cancer by exploring RNAseq data sets available in The Cancer Genome Atlas (https://cancergenome.nih.gov/), and thereafter whether there was any correlation with E2F1 and PRMT5 expression. The MECOM V7 transcript variant was present at increased levels in cervical, colon and ovarian cancer compared to the normal tissue control where, importantly, its level coincided with the expression of E2F1, PRMT5 and MECOM V7 (FIG. 11). These results highlight the role of PRMT5 and p100/TSN-E2F1 in regulating alternative splicing of SENP7 and MECOM RNA, and further suggest that it is relevant to clinical disease.

### Biological Consequence of Alternative Splicing for E2F1 Activity

We wanted to understand the functional significance of alternative splicing directed by meR-E2F1 and p100/TSN for the E2F pathway. To this end, we decided to pursue SENP7 as previous studies had highlighted the role of SENP7 deSUMOylase in the control of HP1, an established repressor of E2F transcriptional activity (24, 25) and a known target for deSUMOylation by SENP7 (20). We assessed whether the SENP7 V5 RNA variant, which selectively interacts with p100/TSN-E2F1 and is dependent on PRMT5 activity, can influence E2F activity. We did this by measuring HP1a and SUMO ChIP activity on the p73 promoter, an established E2F target gene (26). Treating cells with EPZ015666 (which down-regulates the SENP7 V5 RNA variant; FIG. 4D) caused an increase in chromatin-associated SUMOylation on the p73 promoter, which coincided with reduced levels of transcription (FIG. 6A and FIGS. 10F and G). Moreover, the increased chromatin SUMOylation reflected an increased association of HP1a (FIG. 6B). Mechanistically, silencing SENP7 with siRNA caused increased levels of chromatin-associated HP1a (FIG. 6C); a similar effect was observed upon silencing E2F1 (FIG. 6D), thus connecting chromatin SUMOylation to E2F1 activity. We subsequently addressed the specific role of the meR-E2F1 associated spliced RNA variant by expressing SENP7 V5 in cells and measuring the effect on HP1a ChIP activity. Expressing the V5 variant, and the resulting SENP7 protein, decreased the level of HP1a ChIP activity (FIG. 6E). Most significantly, the reduced HP1a ChIP activity coincided with increased transcriptional activity of E2F target genes (FIG. 6F). These results suggest that the V5 variant, derived from an E2F1-dependent alternative splicing effect on SENP7, has a functional consequence on the E2F pathway.

### Discussion

The work described here provides new mechanistic insights into the processes affected by arginine methylation of E2F1, and relates the information to the fundamental properties of the E2F pathway. We found that the methylation mark impacts not only on the repertoire of genes transcriptionally regulated by E2F1, but most importantly enables E2F1 to exert control over alternative RNA splicing of a large group of E2F target genes that otherwise are poor E2F transcriptional targets. We suggest therefore that the methylation mark extends the regulatory impact of E2F1 on gene expression, from one where transcriptional control is the principal level of control, to another where alternative RNA splicing is the predominate process. This pathway provides a mechanism whereby E2F1 can extend its influence to genes which otherwise would be poor transcription targets for E2F1. The meR mark thus widens the genomic landscape under E2F1 control.

We found that components of the splicing machinery associate with E2F1, and that a diverse array of RNAs, mostly derived from E2F target genes, are subject to alternative splicing control in an E2F1-dependent fashion. Moreover, by reading the symR mark on E2F1, p100/TSN recruits an extensive group of RNAs to E2F1, many of which represent alternatively spliced variants. It is known that p100/TSN functions in snRNP assembly, and hence is involved with pre-mRNA splicing (27), and it is consistent with this observation that we identified snRNAs associated with E2F1 that were dependent on PRMT5 activity and E2F1 methylation. This highlights a possible mechanism whereby E2F1 can engage with the splicing machinery to influence the splicing process (FIG. 6G).

Our results make the interesting suggestion that there is a broad division of E2F target genes into two groups: one group regulated through the classical E2F pathway mechanism of transcriptional control and the other consisting of genes which are generally poor E2F transcription targets, where regulation occurs principally through alternative RNA splicing. Reflecting on the biological properties of E2F1, we reason that this broad division into two mechanisms for controlling gene expression could have biological significance in mediating the outcome of E2F1 activity. This is because alternative RNA splicing provides the cell with a great deal of flexibility in protein function, and thus may be relevant in physiological situations where the transcriptional role of E2F1 is compromised.

The analysis of alternative RNA splicing of the SENP7 gene supports the importance of alternative splicing for E2F1 function. Thus, manipulating the expression level of V5 (the SENP7 RNA variant dependent on PRMT5 and E2F1 activity identified in the E2F1 RIP-seq) found that it was an efficient regulator of E2F target gene transcription, most likely through altering the repressive effect of HP1 a on E2F target gene activity. It appears therefore that the ability of E2F1 to impact on alternative RNA splicing has significant functional consequences on E2F1 activity.

In conclusion, our study has revealed an unexpected mechanism whereby arginine methylation widens the regulatory impact of E2F1, from its classical mechanism of transcriptional control to one where alternative RNA splicing is the predominate level of regulation. The reader-writer interplay which is dependent on the meR mark endows E2F1 with a new regulatory RNA splicing mechanism which extends its genomic influence. The meR mark thus expands the repertoire of genomic landscape under E2F control.

### Materials and Methods

### Cell Line Generation

HA-tagged wild-type, the arginine to lysine 111/113 mutant E2F1 (KK), and the arginine to lysine 109 (R109K) constructs have been described previously (11). These were sub-cloned into a pTRE2-hyg expression vector (Clontech) and transfected into parental Tet-On U2OS cells (Clontech; RRID: CVCL_V335) to generate inducible, stable cell lines. These cells were selected in Dulbecco's modified Eagle medium (Sigma) supplemented with 10% (v/v) FBS, penicillin/streptomycin, 100 μg/ml G418 (Santa Cruz Biotechnology) and 150 μg/ml hygromycin B (Toku-E). For all experiments 1 μg/ml doxycycline was used to induce protein expression for 24 h prior to harvest. E2F1 and TSN CRISPR cells were generated as per the protocol described (28), and cultured in DMEM containing 10% (v/v) FBS and penicillin/streptomycin. All cell lines were tested for Mycoplasma contamination prior to use.

### Plasmid/siRNA Transfection

HA-tagged wild type E2F1, E2F1-KK and E2F1 R109K plasmids have been described previously (11). HA-tagged E2F1 L132E and R166H constructs were generated from wild-type HA-E2F1 using a site-directed mutagenesis kit (Stratagene). Flag-tagged SENP7 V5 was generated by sub-cloning from an ORF shuttle clone (OCAAo5051G027D; Source Bioscience) using primers targeting the start and stop codons (flanked with NotI and SalI restriction sites respectively). The PCR product was purified using a PCR purification kit (Qiagen) and digested with the required enzymes (Promega) for 1 h. The digested DNA was gel purified using a Gel Extraction kit (Qiagen) and ligated into the p3×Flag-CMV 7.1 vector (Sigma).

Plasmid transfections were performed for 48 h using Genejuice transfection reagent (Novagen) as per the manufacturer's instructions. RNA interference was performed with 25 nM siRNA for 72 h using Oligofectamine transfection reagent (Invitrogen) as per the manufacturer's instructions. Sequences for siRNA are as follows:

non-targeting control
[SEQ ID No. 9]
(5′-AGCUGACCCUGAAGUUCUU-3′),
E2F1
[SEQ ID No. 7]
(5′-CUCCUCGCAGAUCGUCAUCUU-3′),
p100-TSN
[SEQ ID No. 10]
(5′-AAGGAGCGAUCUGCUAGCUAC-3′),
or
SENP7
[SEQ ID No. 11]
(5′-GAAGUAAGACAGUAGAUGA-3′).

### Immunoblotting and Antibodies

For immunoblots, cells were harvested in RIPA buffer (50 mM Tris-HCl pH 8, 150 mM NaCl, 1% Igepal CA-630 [v/v], 0.5% sodium deoxycholate [w/v], 0.1% SDS [v/v], 0.2 mM sodium orthovanadate and protease inhibitor cocktails) and incubated on ice for 30 mins prior to SDS-PAGE. The following antibodies were used in immunoblots: p100/TSN (A302-883A; Bethyl Laboraties; RRID: AB_10631268), E2F1 (C20; Santa Cruz; RRID: AB_631394), E2F1 (A300-766A, Bethyl Laboratories; RRID: AB_2096774), HA (16B12; Covance; RRID: AB_10063630), FLAG (M2; Sigma; RRID: AB_262044), R-Actin (AC-74; Sigma; RRID: AB_476697), H4R3me2s (ab5823; Abcam; RRID: AB_10562795), Histone H4 (ab10158; Abcam; RRID: AB_296888) and SENP7 (donated by R. Hay, University of Dundee, UK)

### RNA Isolation and QPCR

RNA was isolated from cells using TRIzol (Thermo Fisher) according to the manufacturer's instructions. 1 μg of total RNA was used for cDNA synthesis. For standard mRNA analysis oligo(d)T₂₀ (Invitrogen) was added. For splice variant analysis, RNA was DNAse treated (Sigma-Aldrich) prior to cDNA synthesis using random hexamers (Invitrogen). M-MLV reverse transcriptase (Promega) was used as per the manufacturer's instructions. qRT-PCR was carried out in triplicate using the indicated primer pairs and Brilliant III SYBR Green QPCR Master Mix (Stratagene) on an MX3005P (Agilent) QPCR instrument. Results were expressed as average (mean) fold change compared to control treatments using the ΔΔCt method from three biological repeat samples. GAPDH or actin primer sets were used as an internal calibrator. Error bars represent standard error unless otherwise indicated.

### Chromatin Immunoprecipitation (ChIP)

ChIP was performed as described previously [(29) or (30)]. Antibodies used for immunoprecipitation were as follows: anti-E2F1 (C-20), anti-HA (16B12), anti-HP1α (NB110-40623, Novus Biologicals; RRID: AB_714949), anti-SUMO2/3 (8A2; Abcam; RRID: AB_1658424), non-specific rabbit or mouse IgG. The recovered DNA was analysed in triplicate by real-time QPCR as described (30, 31) on a MX3005P QPCR system using Brilliant III SYBR Green QPCR Master Mix according to the manufacturer's instructions. Results were expressed as average (mean) fold change compared to IgG control treatments using the ΔΔCt method from triplicate biological repeat samples. Alternatively, a standard curve was generated to calculate ChIP/input signals that were subsequently used to generate fold change values compared to IgG control. Error bars represent standard error unless otherwise indicated.

### RNA Immunoprecipitation (RIP)

Cells were washed once with PBS before UV cross-linking at 900 mJ/cm² using a Stratalinker (Stratagene). RIP lysis buffer (50 mM Tris-HCl pH 8.0, 150 mM NaCl, 1 mM MgCl₂, 10% glycerol [v/v], 1% NP-40 [v/v], 1 mM DTT, 0.2 mM sodium orthovanadate and protease inhibitor cocktails) was added directly to the plate, on ice. The lysate was agitated at 4° C. for 10 mins before sample clarification at 13,000 rpm. For protein samples, 5% of inputs were taken and boiled in SDS-loading buffer. For RNA samples, 10% of inputs were taken and 10 μg proteinase K added for thirty minutes at 37° C. before addition of TRIzol and RNA isolation. The rest of the lysate was pre-cleared using pre-blocked protein A/G agarose beads, 1 μg of non-specific IgG (Jackson ImmunoResearch) and 0.1 mg/ml heparin for one hour at 4° C. The pre-cleared lysate was added to a fresh tube with 1 μg of non-specific IgG, or specific antibody (E2F1, C-20, Santa Cruz; RRID: AB_631394) for 1 h with rotation. Protein A/G beads were then added for a further hour. The beads were washed 4 times in RIP lysis buffer and resuspended in 400 μl RIP lysis buffer. This was separated into two fractions—one for protein isolation and the other for RNA extraction. For protein isolation, beads were dried and resuspended in SDS-loading buffer before boiling. For RNA extraction, an equal amount of RIP extraction buffer (350 mM NaCl, 10 mM Tris-HCl pH 7.5, 10 mM EDTA, 0.1% SDS [w/v], 7 M urea) was added to the fraction, along with 15 μg proteinase K, and incubated at 37° C. for 30 mins before RNA purification using Trizol. RNA was DNase treated prior to first-strand cDNA synthesis using random hexamers and M-MLV reverse transcriptase.

### RNA-Sequencing (RNA-seq)

Wild-type, E2F1-KK, or E2F1-R109K expression was induced in U2OS-Tet-ON cells for 24 h before isolating the RNA using TRIzol. mRNA was subsequently enriched from three biological replicates using the NEBNext Poly(A) mRNA Magnetic Isolation Module (NEB) as per the manufacturer's instructions. cDNA libraries were made using NEBNext® Ultra™ Directional RNA Library Prep Kit for Illumina® (NEB). Sequencing was carried out on an Illumina NextSeq platform.

### RNA-Seq Data Analysis

FASTQ files for pTRE, WT, KK and R109K samples in three biological replicates were trimmed to remove adapters and low-quality bases with TrimGalore v.0.4.3 (http://www.bioinformatics.babraham.ac.uk/projects/trim_galore/). The trimmed reads were aligned to the human reference genome (build hg19) with STAR aligner v.2.5.1 (32) with two mismatches allowed. Differential gene expression analysis was done with DESeq2 R Bioconductor package v.1.16.1 (33), using read counts data provided by the aligner. Genes were considered differentially expressed if the adjusted P-value, calculated using the Benjamini-Hochberg method in order to minimise the false discovery rate (FDR), was less than 0.01 and the change in expression level was greater than 2-fold. Differential splicing analysis, 4J calculation and splicing events statistics was done with rMATS turbo package v4.0.1 (17). The FDR threshold for differential PSI was chosen to be 0.01. The GO enrichment analysis was done with MetaCore software suite (Clarivate Analytics, v.6.33-69110) to reveal biological processes over-represented in differentially spliced gene sets. P-values for GO enrichment analysis were calculated using the formula for hypergeometric distribution, reflecting the probability for a GO term to arise by chance. Statistically enriched terms were identified using a threshold FDR of 3%. Clustering of GO:BP terms was performed using R Bioconductor goseq package (v.1.30) and annotations provided in org.Hs.eg.db (v.3.5.0) and GO.db (v.3.5) packages. Gene expression data have been deposited in NCBI's Gene Expression Omnibus and are accessible through GEO Series accession number GSE111961.

### RIP-Sequencing (RIP-seq)

An E2F1 RIP was performed as described above, from samples treated for 72 h with non-targeting siRNA, or siRNA against TSN. An E2F1 siRNA condition was also included for the RIP-seq as a control to monitor for specificity of the RNA species identified. Following RNA extraction and DNAse treatment, equal volumes of sample were taken and underwent ribodepletion using GeneRead rDNA Depletion kit (Qiagen). Libraries were prepared using NEBNext Ultra Directional RNA library Prep kit for Illumina® (NEB). The library was sequenced on an Illumina NextSeq and bioinformatics analysis was carried out (see below).

### RIP-Seq Data Analysis

FASTQ files for two biological replicates in each condition were trimmed as described above. The reads were aligned to the human genome build hg19 by gsnap aligner v.2017-04-21 with two mismatches allowed (34). The RIPSeq analysis was performed with RIPSeeker R package v.1.18.0 (35) with the parameters as follows: uniqueHit=TRUE, assignMultihits=TRUE, rerunWithDisambiguatedMultihits=TRUE and automatic bin size selection. EnsEMBL bioMart build 75 was used for functional annotation of the RIPSeq results. RNA species significantly enriched (p adj value threshold <0.05) above the siE2F1 control RIP were recorded in Table S4. RIP sequencing data have been deposited in NCBI's Gene Expression Omnibus and are accessible through GEO Series accession number GSE111961.

### Gene Set Analysis

Gene set analysis was performed with the piano R package (v.1.8.2) using the Mean method (36), with 1000 permutations and with minimum and maximum gene sets of 15 and 500, respectively, against the 50 hallmark (h) gene sets from the MSigDB (v.6.1). Resulting gene sets with a nominal P value of 0.05 were considered significant. Distinct directional network maps were visualised with the piano R package.

### Xena Browser Functional Genomics Analysis

For the analysis of E2F1, PRMT5, MECOM V7 and total MECOM expression levels in human cancers, Xena browser (University of California) was used (https://xena.ucsc.edu/). The TCGA TARGET GTEx dataset was selected, which contained transcript expression data from the Cancer Genome Atlas (TCGA, cancer tissue) and Genotype-Tissue Expression (GTEx, healthy tissue) samples. Cervical, colon and ovarian cancers were selected alongside their respective healthy tissue, and were categorised according to their E2F1 gene expression. Information on PRMT5 and MECOM gene expression was also displayed. MECOM V7 transcript was identified using the ENSEMBL transcript ID.

### Immunofluorescence

U2OS cells (HTB-96, ATCC; RRID: CVCL_0042) were plated on coverslips and transfected for 48 h with the indicated plasmids, or U2OS-Tet-ON cells were induced to express wild-type E2F1, E2F1-KK, or E2F1-R109K for 24 h as appropriate. Cells were fixed for 15 mins in 4% paraformaldehyde/PBS and permeabilised for 15 mins in 0.5% Triton X-100/PBS. Coverslips were incubated with primary antibody for 1 h, washed 5 times and then incubated with Alexa Fluor-488 conjugated secondary antibody (Thermo Fisher; RRID: AB_141607) for 1 h. Coverslips were washed again before mounting on glass slides using Vectashield mounting medium with DAPI (Vectorlabs). Proteins were visualised on a BX60 fluorescence microscope (Olympus) fitted with a Hamamatsu C4742-95 camera, and analysed with Openlab 5 software (Improvision).

### Flow Cytometry

Wild-type, E2F1-KK, or E2F1-R109K mutant U2OS-Tet-ON cells were induced with doxycycline for 24 h before addition of fresh media containing 20 μM etoposide and doxycycline for 48 h. Then, cells were fixed and stained with propidium iodide for cell cycle analysis, as described previously (30).

### Clonogenic Assay

1000 cells were seeded into 6-well plates in triplicate and left to settle overnight. Doxycycline was added the following morning to induce protein expression and was topped up every 72 h over the 10 day period. After 10 days, cells were washed twice in PBS before fixation in ice cold methanol for 20 mins. Methanol was removed and 0.5% crystal violet stain was added for 10 mins. The colonies were washed thoroughly in water and left to dry prior to counting.

### Luciferase Reporter Assays

U2OS cells were transfected with 500 ng p73-luciferase or CDC6-luciferase plasmids, along with 500 ng β-galactosidase and 2 μg of p3×Flag-CMV SENP7 V5 or empty vector for 48 h. Cells extracts were then prepared in Reporter Lysis Buffer (Promega) and combined with luciferase reagent (Promega) for signal detection on a Microlumat Plus LB 96V luminometer (Berthold Technologies). Alternatively, extract was mixed with β-galactosidase buffer (200 mM Na₂PO₄, pH 7.3; 2 mM MgCl₂; 100 mM β-mercaptoethanol; 1.33 mg/ml ONPG) and incubated at 37° C. prior to absorbance monitoring (415 nm) on a Sunrise microplate reader (Tecan). Reporter activity was determined from triplicate technical repeats as luciferase/β-galactosidase reading, and expressed as fold induction compared to empty vector expressing cells. Displayed are average (mean) fold changes with standard error from two biological repeat experiments.

### Statistical Analysis

Statistical analyses were performed using two-tailed, unpaired Student's t-test with Excel software (Microsoft). Data are shown as means with standard error displayed. P-values are indicated as *P<0.05 or **P<0.005.

### Data Availability

RNA-seq and RIP-seq data sets that support the findings of this study have been deposited in NCBI's Gene Expression Omnibus with the accession code GSE111961.

### REFERENCES

1. R. Di Fiore, A. D'Anneo, G. Tesoriere, R. Vento, RB1 in cancer: different mechanisms of RB1 inactivation and alterations of pRb pathway in tumorigenesis. Journal of cellular physiology 228: 1676-1687 (2013)
2. S. Munro, S. M. Carr, N. B. La Thangue, Diversity within the pRb pathway: is there a code of conduct? oncogene 31: 4343-4352 (2012)
3. A. P. Roworth, F. Ghari, N. B. La Thangue, To live or let die—complexity within the E2F1 pathway. Molecular & Cellular Oncology 2: e970480 (2015)
4. L. Wu, C. Timmers, B. Maiti, H. I. Saavedra, L. Sang, G. T. Chong, F. Nuckolls, P. Giangrande, F. A. Wright, S. J. Field, M. E. Greenberg, S. Orkin, J. R. Nevins, M. L. Robinson, G. Leone, The E2F1-3 transcription factors are essential for cellular proliferation. Nature 414: 457-462 (2001)
5. G. Yao, T. J. Lee, S. Mori, J. R. Nevins, L. You, A bistable Rb-E2F switch underlies the restriction point. Nature Cell Biology 10: 476-482 (2008)
6. S. J. Field, F. Y. Tsai, F. Kuo, A. M. Zubiaga, W. G. Kaelin, D. M. Livingston, S. H. Orkin, M. E. Greenberg, E2F-1 Functions in mice to promote apoptosis and suppress proliferation. Cell 85:549-561 (1996)
7. E. Lazzerini Denchi, K. Helin, E2F1 is crucial for E2F-dependent apoptosis. EMBO reports 6: 661-668 (2005)
8. A. M. Pierce, R. Schneider-Broussard, I. B. Gimenez-Conti, J. L. Russell, C. J. Conti, D. G. Johnson, E2F1 has both oncogenic and tumor-suppressive properties in a transgenic model. Molecular and cellular biology 19: 6408-6414 (1999)
9. S. M. Carr, A. P. Roworth, C. Chan, N. B. La Thangue, Post-translational control of transcription factors: methylation ranks highly. The FEBS journal 282: 4450-4465 (2015)
10. Y. Yang, M. T. Bedford, Protein arginine methyltransferases and cancer. Nat Rev Cancer 13: 37-50 (2013)
11. E-C. Cho, S. Zheng, S. Munro, G. Liu, S. M. Carr, J. Moehlenbrink, Y-C. Lu, L. Stimson, O. Khan, R. Konietzny, J. McGouran, A. S. Coutts, B. Kessler, D. J. Kerr, N. B. La Thangue, Arginine methylation controls growth regulation by E2F-1. EMBO journal 31: 1785-1797 (2012)
12. S. Zheng, J. Moehlenbrink, Y-C. Lu, L-P. Zalmas, C. A. Sagum, S. M. Carr, J. McGouran, L. Alexander, O. Fedorov, S. Munro, B. Kessler, M. T. Bedford, Q. Yu, N. B. La Thangue, Arginine Methylation-Dependent Reader-Writer Interplay Governs Growth Control by E2F-1. Molecular cell 52: 37-51 (2013)
13. C. Su, C. Zhang, A. Tecle, X. Fu, J. He, J. Song, W. Zhang, X. Sun, Y. Ren, O. Silvennoinen, Z. Yao, X. Yang, M. Wei, J. Yang, Tudor staphylococcal nuclease (Tudor-SN), a novel regulator facilitating G1/S phase transition, acting as a co-activator of E2F-1 in cell cycle regulation. The Journal of biological chemistry 290: 7208-7220 (2015)
14. M. Bieda, X. Xu, M. A. Singer, R. Green, P. J. Farnham, Unbiased location analysis of E2F1-binding sites suggests a widespread role for E2F1 in the human genome. Genome Research 16: 595-605 (2006)
15. H. Müller, A. P. Bracken, R. Vernell, M. C. Moroni, F. Christians, E. Grassilli, E. Prosperini, E. Vigo E, J. D. Oliner, K. Helin, E2Fs regulate the expression of genes involved in differentiation, development, proliferation, and apoptosis. Genes & development 15: 267-285 (2001)
16. E. Gutierrez-Beltran, T. V. Denisenko, B. Zhivotovsky, P. V. Bozhkov, Tudor staphylococcal nuclease: biochemistry and functions. Cell Death & Differentiation 23: 1739-1748 (2016)
17. S. Shen, J. W. Park, Z. X. Lu, L. Lin, M. D. Henry, Y. N. Wu, Q. Zhou, Y. Xing, rMATS: robust and flexible detection of differential alternative splicing from replicate RNA-Seq data. Proc Natl Acad Sci USA 111: E5593-5601 (2014)
18. S. Valadkhan, L. S. Gunawardane, Role of small nuclear RNAs in eukaryotic gene expression. Essays Biochem 54: 79-90 (2013)
19. E. Chan-Penebre, K. G. Kuplast, C. R. Majer, P. A. Boriack-Sjodin, T. J. Wigle, L. D. Johnston, N. Rioux, M. J. Munchhof, L. Jin, S. L. Jacques, K. A. West, T. Lingaraj, K. Stickland, S. A. Ribich, A. Raimondi, M. P. Scott, N. J. Waters, R. M. Pollock, J. J. Smith, O. Barbash, M. Pappalardi, T. F. Ho, K. Nurse, K. P. Oza, K. T. Gallagher, R. Kruger, M. P. Moyer, R. A. Copeland, R. Chesworth, K. W. Duncan, A selective inhibitor of PRMT5 with in vivo and in vitro potency in MCL models. Nat Chem Biol 11: 432-437 (2015)
20. T. Bawa-Khalfe, L-S. Lu, Y. Zuo, C. Huang, R. Dere, F-M. Lin, E. T. H. Yeh, Differential expression of SUMO-specific protease 7 variants regulates epithelial-mesenchymal transition. Proceedings of the National Academy of Sciences 109: 17466-17471 (2012)
21. A. J. Garvin, R. M. Densham, S. A. Blair-Reid, K. M. Pratt, H. R. Stone, D. Weekes, K. J. Lawrence, J. R. Morris, The deSUMOylase SENP7 promotes chromatin relaxation for homologous recombination DNA repair. EMBO Rep 14: 975-983 (2013)
22. R. González-Prieto, S. A. Cuijpers, R. Kumar, I. A. Hendriks, A. C. Vertegaal, c-Myc is targeted to the proteasome for degradation in a SUMOylation-dependent manner, regulated by PIAS1, SENP7 and RNF4. Cell Cycle 14:1859-1872 (2015)
23. X. Yuan, X. Wang, K. Bi, G. Jiang, The role of EVI-1 in normal hematopoiesis and myeloid malignancies. International journal of oncology 47: 2028-2036 (2015)
24. M. Narita, S. Nunez, E. Heard, M. Narita, A. W. Lin, S. A. Hearn, D. L. Spector, G. J. Hannon, S. W. Lowe, Rb-mediated heterochromatin formation and silencing of E2F target genes during cellular senescence. Cell 113: 703-716 (2003)
25. I. Panteleeva, S. Boutillier, V. See, D. G. Spiller, C. Rouaux, G. Almouzni, D. Bailly, C. Maison, H. C. Lai, J. P. Loeffler, A. L. Boutillier, HP1alpha guides neuronal fate by timing E2F-targeted genes silencing during terminal differentiation. EMBO J 26: 3616-3628 (2007)
26. N. Pediconi, A. Ianari, A. Costanzo, L. Belloni, R. Gallo, L. Cimino, A. Porcellini, I. Screpanti, C. Balsano, E. Alesse, A. Gulino, M. Levrero, Differential regulation of E2F1 apoptotic target genes in response to DNA damage. Nature Cell Biology 5: 552-558 (2003)
27. J. Yang, T. Valineva, J. Hong, T. Bu, Z. Yao, O. N. Jensen, M. J. Frilander, O. Silvennoinen, Transcriptional co-activator protein p100 interacts with snRNP proteins and facilitates the assembly of the spliceosome. Nucleic acids research 35: 4485-4494 (2007)
28. F. A. Ran, P. D. Hsu, J. Wright, V. Agarwala, D. A. Scott, F. Zhang, Genome engineering using the CRISPR-Cas9 system. Nat Protocols 8: 2281-2308 (2013)
29. L-P. Zalmas, X. Zhao, A. L. Graham, R. Fisher, C. Reilly, A. S. Coutts, N. B. La Thangue, DNA-damage response control of E2F7 and E2F8. EMBO Reports 9: 252-259 (2008)
30. S. M. Carr, S. Munro, C. A. Sagum, O. Fedorov, M. T. Bedford, N. B. La Thangue, Tudor-domain protein PHF20L1 reads lysine methylated retinoblastoma tumour suppressor protein. Cell Death & Differentiation 24: 2139-2149 (2017)
31. M. Jansson, S. T. Durant, E-C. Cho, S. Sheahan, M. Edelmann, B. Kessler, N. B. La Thangue, Arginine methylation regulates the p53 response. Nature Cell Biology 10: 1431-1439 (2008)
32. A. Dobin, T. R. Gingeras, Optimizing RNA-Seq Mapping with STAR. Methods in Molecular Biology 1415: 245-262 (2016)
33. M. I. Love, W. Huber, S. Anders, Moderated estimation of fold change and dispersion for RNA-seq data with DESeq2. Genome Biology 15: 550 (2014)
34. T. D. Wu, S. Nacu, Fast and SNP-tolerant detection of complex variants and splicing in short reads. Bioinformatics 26: 873-881 (2010)
35. Y. Li, D. Y. Zhao, J. F. Greenblatt, Z. Zhang, RIPSeeker: a statistical package for identifying protein-associated transcripts from RIP-seq experiments. Nucleic Acids Res 41: e94 (2013)
36. B. Efron, R. Tibshirani, On testing the significance of sets of genes. The Annals of Applied Statistics 1: 107-129 (2007)
37. WO-A-2018/154104 to JANSSEN PHARMACEUTICA NV “Use Of Biomarkers In Identifying Cancer Patients That Will Be Responsive To Treatment With A PRMT5 Inhibitor”
38. WO-A-2018/100535 to GLAXOSMITHKLINE INTELLECTUAL PROPERTY DEVELOPMENT LIMITED “Combination Therapy”
39. WO-A-2018/183544 to DANA-FARBER CANCER INSTITUTE, INC. “Method for Identification of Retained Intron Tumor Neoantigens From Patient Transcriptome”
40. Braun, David A, and Catherine J Wu. “Antigen Discovery and Therapeutic Targeting in Hematologic Malignancies.” Cancer journal (Sudbury, Mass.) vol. 23,2 (2017): 115-124.

1. An agent for use in a method of treating cancer by modifying the neoantigen profile of at least one cancer cell.

2. A method of treating a cancer in a patient, comprising treating the patient with an agent that modifies the neoantigen profile of the cancer.

3. An agent for use of claim 1 or a method of claim 2, wherein the modifying is increasing the number of neoantigens present on the cancer cell.

4. A method or agent for use of any preceding claim, wherein the agent increases intron retention in a cancer cell or increases the stability of one or more retained introns in a cancer cell.

5. A method or agent for use of any preceding claim, wherein the agent alters the arginine methylation state of a transcription regulator, optionally an E2F protein.

6. A method or agent for use of any preceding claim, wherein the agent is an E2F1 methylation inhibitor.

7. A method or agent for use of any preceding claim, wherein the agent is a protein arginine N-methyltransferase 5 (PRMT5) inhibitor.

8. A method or agent for use according to any preceding claim, wherein the agent is an antisense molecule, an shRNA, an siRNA, a small molecule with a molecular weight less than 750 Da, a protein, an antibody or an antigen-binding fragment of an antibody.

9. A method or agent for use of any preceding claim, wherein the method of treatment comprises the steps of:
(a) administering the agent to the cancer patient;
(b) identifying retained introns in RNA transcripts from one or more cancer cells from the cancer patient to whom the agent has been administered, optionally from a tumour biopsy;
(c) identifying one more neo-antigens expressed by the retained introns; and
(d) administering to the patient one or more of the identified neo-antigens.

10. A method or agent for use according to claim 9, further comprising the steps of:
(e) isolating T cells from a tumour microenvironment in the patient;
(f) expanding in vitro isolated T cells that have affinity for the one or more administered neoantigens, optionally wherein the T cells are CD8+ CTLs; and
(g) administering to the patient the expanded T cells.

11. A method or agent for use of any preceding claim, as part of a combination therapy.

12. A method or agent for use of claim 11, wherein the combination therapy comprises a chemotherapeutic agent or an immunotherapy.

13. A method or agent for use of claim 12, wherein the immunotherapy is a checkpoint inhibitor.

14. A method or agent for use of claim 12 or claim 13, wherein the immunotherapy is an anti-PD-1 antibody, an anti-PD-L1 antibody, an anti-CTLA-4 antibody, an anti-LAG-3 antibody, an anti-CTLA-4 antibody, a CAR-T cell or a CAR-NK cell.

15. A method or agent for use of any preceding claim, wherein the cancer is:
an oesophageal, pancreatic, gastric or hepatic cancer;
a carcinoma, optionally a colon carcinoma, an oesophageal carcinoma or a hepatocellular carcinoma; or
an adenocarcinoma optionally of the colon, pancreas or stomach.

16. A method of determining whether a patient will respond favourably to cancer immunotherapy, comprising
(a) administering to the cancer patient an agent to modify the neoantigen profile of a cancer cell in the patient; and
(b) identifying the patient as likely to benefit from immunotherapy if one or more introns are identified in RNA transcripts from a cancer cell from the cancer patient to whom the agent has been administered, optionally wherein the RNA transcripts are identified from a tumour biopsy.

17. An in vitro method for identifying an agent useful in treating cancer, comprising contacting the agent with a cell and assessing whether the level of retained introns in RNA transcripts increases following contact, wherein an increase in retained introns indicates that the agent is useful in treating cancer.

18. An in vitro method of increasing neoepitope expression in a cell, comprising contacting the cell with an agent that increases intron retention in a cell, optionally wherein the agent that increases intron retention in a cell reduces methylation of an E2F protein.

19. An in vitro method for identifying a neoantigen for use in treating cancer, comprising the steps of:
(a) identifying retained introns in RNA transcripts from a cancer cell from a patient;
(b) identifying one more neo-antigens expressed by the retained introns.

20. An in vitro method according to claim 19, further comprising the step of producing the one or more neo-antigens and optionally formulating the one or more neo-antigens into a pharmaceutical composition.

21. An in vitro method according to claim 19 or claim 20, wherein the cancer cell in step (a) is from a patient to whom an agent has been administered to modify the cancer neoantigen profile.

22. A pharmaceutical composition comprising an agent capable of modifying the neoantigen profile of a cancer cell.

23. A pharmaceutical composition comprising one or more neoantigens, wherein the one or more neoantigens are encoded by RNA comprising one or more retained introns.

24. A pharmaceutical composition according to claim 23, wherein at least one neoantigen is prepared for a pre-determined patient or cancer, optionally wherein the neoantigen formulation is prepared individually for the patient.

25. A pharmaceutical composition according to claim 23 or 24, comprising an adjuvant.

26. An ex vivo composition comprising RNA sequences encoding one or more neoantigens, wherein each RNA sequence comprises at least one retained intron per neoantigen.

27. An ex vivo composition according to claim 26, wherein the RNA sequences are human and optionally comprising one or more non-human reagents optionally selected from a non-human DNA or RNA polymerase molecule.

28. An immune cell engineered to express one or more receptors that specifically binds to one or more neoantigens encoded by RNA containing at least one retained intron, optionally wherein the retained intron has been identified by the method of claim 19.

29. An immune cell according to claim 28, which is a T cell or NK cell, optionally a CAR-T cell or a CAR-NK cell.

30. A population of immune cells according to claim 28 or 29, optionally comprising at least two different cells with affinity for different neoantigens.

31. An autologous cell therapy product comprising an expanded T cell population having T cell receptors with affinity for a neoantigen present on a cancer cell, wherein the population was expanded from one or more T cells sampled from a tumour microenvironment.

32. A neoantigen or combination of neoantigens produced by the method of claim 19 or claim 20.


SEQUENCE LISTING (14 sequences)

SEQ ID NO: 1 - protein, 637 aa, Homo sapiens
MAAMAVGGAGGSRVSSGRDLNCVPEIADTLGAVAKQGFDFLCMPVFHPRFKREFIQEPAKNRPGPQTRSDLLLSGRDWNTLIVGKLSPWIRPDSKVEKIRRNSEAAMLQELNFGAYLGLPAFLLPLNQEDNTNLARVLTNHIHTGHHSSMFWMRVPLVAPEDLRDDIIENAPTTHTEEYSGEEKTWMWWHNFRTLCDYSKRIAVALEIGADLPSNHVIDRWLGEPIKAAILPTSIFLTNKKGFPVLSKMHQRLIFRLLKLEVQFIITGTNHHSEKEFCSYLQYLEYLSQNRPPPNAYELFAKGYEDYLQSPLQPLMDNLESQTYEVFEKDPIKYSQYQQAIYKCLLDRVPEEEKDTNVQVLMVLGAGRGPLVNASLRAAKQADRRIKLYAVEKNPNAVVTLENWQFEEWGSQVTVVSSDMREWVAPEKADIIVSELLGSFADNELSPECLDGAQHFLKDDGVSIPGEYTSFLAPISSSKLYNEVRACREKDRDPEAQFEMPYVVRLHNFHQLSAPQPCFTFSHPNRDPMIDNNRYCTLEFPVEVNTVLHGFAGYFETVLYQDITLSIRPETHSPGMFSWFPILFPIKQPITVREGQTICVRFWRCSNSKKVWYEWAVTAPVCSAIHNPTGRSYTIGL

SEQ ID NO: 2 - RNA, 19 nt, Homo sapiens
ccgcuauugcaccuuggaa

SEQ ID NO: 3 - RNA, 19 nt, Homo sapiens
caacagagauccuaugauu

SEQ ID NO: 4 - protein, 5 aa, Homo sapiens
  MOD_RES (3)..(3): METHYLATION
  MOD_RES (5)..(5): METHYLATION
RGRGR

SEQ ID NO: 5 - protein, 17 aa, Homo sapiens
  MOD_RES (10)..(10): METHYLATION
  MOD_RES (12)..(12): METHYLATION
CESSGPARGRGRHPGKG

SEQ ID NO: 6 - protein, 5 aa, Homo sapiens
RGRGR

SEQ ID NO: 7 - RNA, 21 nt, Homo sapiens
cuccucgcagaucgucaucuu

SEQ ID NO: 8 - protein, 437 aa, Homo sapiens
MALAGAPAGGPCAPALEALLGAGALRLLDSSQIVIISAAQDASAPPAPTGPAAPAAGPCDPDLLLFATPQAPRPTPSAPRPALGRPPVKRRLDLETDHQYLAESSGPARGRGRHPGKGVKSPGEKSRYETSLNLTTKRFLELLSHSADGVVDLNWAAEVLKVQKRRIYDITNVLEGIQLIAKKSKNHIQWLGSHTTVGVGGRLEGLTQDLRQLQESEQQLDHLMNICTTQLRLLSEDTDSQRLAYVTCQDLRSIADPAEQMVMVIKAPPETQLQAVDSSENFQISLKSKQGPIDVFLCPEETVGGISPGKTPSQEVTSEEENRATDSATIVSPPPSSPPSSLTTDPSQSLLSLEQEPLLSRMGSLRAPVDEDRLSPLVAADSLLEHVREDFSGLLPEEFISLSPPHEALDYHFGLEEGEGIRDLFDCDFGDLTPLDF

SEQ ID NO: 9 - RNA, 19 nt, Homo sapiens
agcugacccugaaguucuu

SEQ ID NO: 10 - RNA, 21 nt, Homo sapiens
aaggagcgaucugcuagcuac

SEQ ID NO: 11 - RNA, 19 nt, Homo sapiens
gaaguaagacaguagauga

SEQ ID NO: 12 - protein, 12 aa, Homo sapiens
PARGRGRHPGKG

SEQ ID NO: 13 - protein, 12 aa, Homo sapiens
PAKGRGRHPGKG

SEQ ID NO: 14 - protein, 12 aa, Homo sapiens
PARGKGKHPGKG